(19) United States
(12) Patent Application Publication
(10) Pub. No.: US 2026/0174641 A1
(43) Pub. Date: Jun. 25, 2026

(54) Oral Care Mousse Compositions And Products Thereof

(71) Applicant: Colgate-Palmolive Company, New York, NY (US)

(72) Inventors: Mohammad Mansoor AZIZ, Jersey City, NJ (US); Lauren EVANS, Highland Park, NJ (US); Murilo NOGUEIRA NAKAJIMA, Sao Paulo (BR); Shashank Vishwanath POTNIS, Princeton, NJ (US); Sara MORALES AVILES, Mexico City (MX); Baran TEOMAN, Lake Hiawatha, NJ (US); Andrei POTANIN, Hillsborough, NJ (US); Juhi SOLANKI, New Brunswick, NJ (US)

(73) Assignee: Colgate-Palmolive Company, New York, NY (US)

(21) Appl. No.: 19/432,003

(22) Filed: Dec. 23, 2025

Related U.S. Application Data
(60) Provisional application No. 63/738,297, filed on Dec. 23, 2024.

(51) Int. Cl. A61K 8/19; A61Q 11/00

(52) U.S. Cl. CPC A61K 8/19; A61Q 11/00; A61K 2800/28; A61K 2800/30; A61K 2800/48; A61K 2800/592; A61K 2800/596; A61K 2800/87; A61K 2800/92

(57) ABSTRACT

Oral care compositions, particularly oral care mousse compositions, as well as oral care products thereof are disclosed herein. In accordance with one aspect, provided is an oral care product comprising: an oral care package comprising a vessel portion and a nozzle; and an oral care composition disposed in the vessel portion, the oral care composition comprising: from about 10 to about 43 wt. % of an abrasive; a surfactant system comprising: an amphoteric surfactant, and a nonionic surfactant; and a thickening system comprising: a natural gum, and an acrylic acid polymer, wherein the oral care composition has a stress drop of about 18 to about 60% and an adhesive force of about 60 to about 200 gram-force, and wherein all weight percentages are based on the total weight of the oral care composition.

### CROSS-REFERENCE TO RELATED APPLICATION

This application claims the benefit of priority from U.S. Provisional Application No. 63/738,297, filed Dec. 23, 2024, the contents of which are hereby incorporated herein by reference in their entirety.

### BACKGROUND

Numerous oral care compositions have been developed with the aim of providing therapeutic benefits to consumers, such as caries reduction and to treat, prevent or ameliorate various oral diseases and conditions. Improvements in the non-therapeutic aspects of oral care products, such as the development of improved textures, the inclusion of flavors and an improvement in the aesthetic appeal have been devised. Advantages of these formulations include increased compliance by consumers with tooth-cleaning regime, which may result in improved oral and overall health. In view of oral care consumers constantly evolving preferences for oral care products that deliver therapeutic as well as non-therapeutic aspects, there remains a need in the art for dentifrices of aesthetic and gustatory appeal that simultaneously provide therapeutic benefits to the consumer.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to oral care compositions, particularly, oral care mousse compositions, as well as oral care products thereof. In accordance with an aspect of the invention, provided is an oral care product comprising: an oral care package comprising a vessel portion and a cap coupled to the vessel portion, the cap comprising a nozzle; and an oral care composition disposed in the vessel portion, the oral care composition comprising: from about 10 to about 43 wt. % of an abrasive; a surfactant system comprising: an amphoteric surfactant, and a nonionic surfactant; and a thickening system comprising: a natural gum, and an acrylic acid polymer, wherein the oral care composition has a stress drop of about 18 to about 60% and an adhesive force of about 60 to about 200 gram-force, and wherein all weight percentages are based on the total weight of the oral care composition.

According to another aspect of the invention, provided is an oral care composition comprising: from about 10 to about 43 wt. % of an abrasive; a surfactant system comprising: an amphoteric surfactant, and a nonionic surfactant; and a thickening system comprising: a natural gum, and an acrylic acid polymer, wherein the oral care composition has a stress drop of about 18 to about 60% and an adhesive force of about 60 to about 200 gram-force, and wherein all weight percentages are based on the total weight of the oral care composition.

In accordance with a further aspect of the invention, provided is an oral care composition comprising: from about 10 to about 43 wt. % of an abrasive, the abrasive comprising an alkali metal carbonate, an alkali earth metal carbonate, an insoluble phosphate, or a combination of two or more thereof; a surfactant system; a thickening system comprising: a natural gum, and an acrylic acid polymer, wherein the natural gum and the acrylic acid polymer are present in a weight ratio of the natural gum to the polyacrylic acid polymer of about 5:1 to about 1:5; from about 15 to about 44 wt. % of a polyol; and from about 20 to about 50 wt. % of water, wherein the oral care composition has a stress drop of about 18 to about 60% and an adhesive force of about 60 to about 200 gram-force, and wherein the oral care composition is free of propellants and all weight percentages are based on the total weight of the oral care composition. In another aspect, the nozzle architecture provides for mousse formation through a legged aperture (center portion with radial legs) and specific geometry (e.g., leg L/W ratio, leg-area-to-center-area ratio, tip angles) that, when paired with the compositions of the present invention and without a propellant, yields mousse-like nurdles having high foamability and fine, uniform bubbles. In some embodiments, the compositions according to the present invention provides bubbles in sizes of less than 100 μm.

### BRIEF DESCRIPTION OF THE DRAWINGS

Implementation of certain embodiments the present technology will now be described, by way of example only, with reference to the attached figures, wherein:

FIG. 1 is a perspective view of an oral care product in accordance with aspects of the invention;

FIG. 2 is a perspective view of another configuration of the oral care product of FIG. 1;

FIG. 3 is a perspective view of the cap of the oral care product of FIG. 2;

FIG. 4 is a perspective view of the cap of FIGS. 1-3

FIG. 5 is a top view of the cap of FIGS. 1-4;

FIG. 6 is a cross-sectional view of the cap of FIG. 5;

FIG. 7 is a top view of the nozzle of the cap of FIG. 5; and

FIG. 8 is a cross-sectional view of the nozzle of FIG. 5.

FIG. 9 depicts the results of dynamic surface tension analysis on an exemplary composition of the present invention and three comparative compositions.

FIG. 10 depicts data from a foam generation/foam decay evaluation of an exemplary composition of the present invention (Ex. C) and a comparative formula (Comp. Ex. 2).

FIG. 11 depicts data from a foam generation comparison between an exemplary composition of the present invention (Ex. C) and a comparative formula (Comp. Ex. 2).

FIG. 12 depicts the results of an evaluation of the bubbles created by an exemplary composition of the present invention (Ex. C) and a comparative formula (Comp. Ex. 2).

FIG. 13 depicts a nurdle of an exemplary composition of the present invention delivered onto an oral care implement using an exemplary dispenser in accordance with some embodiments of the present invention.

FIG. 14 depicts a front view of an exemplary composition of the present invention delivered onto an oral care implement using an exemplary dispenser in accordance with some embodiments of the present invention.

FIG. 15 depicts a rear view of an exemplary composition of the present invention delivered onto an oral care implement using an exemplary dispenser in accordance with some embodiments of the present invention.

FIG. 16 depicts a right-side view of an exemplary composition of the present invention delivered onto an oral care implement using an exemplary dispenser in accordance with some embodiments of the present invention.

FIG. 17 depicts a left-side view of an exemplary composition of the present invention delivered onto an oral care implement using an exemplary dispenser in accordance with some embodiments of the present invention.

It should be understood that the various aspects are not limited to the arrangements and instrumentality shown in the drawings. Additionally, all drawings are not necessarily to scale. Components numbered and appearing in one figure but appearing un-numbered in other figures are the same unless expressly noted otherwise. A reference herein to a whole figure number which appears in multiple figures bearing the same whole number but with different alphabetical suffixes shall be constructed as a general refer to all of those figures unless expressly noted otherwise.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the oral care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” as referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “−”, “=” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds. Accordingly, as used herein, “substantially free” typically refers to 1 wt. % or less, preferably about 0.5 wt. % or less, preferably about 0.1 wt. % or less, preferably about 0.05 wt. % or less, or preferably about 0.01 wt. % or less, or preferably 0 wt. %, based on the total weight of the oral care composition.

Some of the various categories of components identified may overlap. In such cases where overlap may exist and the oral care composition includes both components (or the composition includes more than two components that overlap), an overlapping compound does not represent more than one component. For example, certain compounds may be characterized as both a sweetener and a polyol. If a particular oral care composition includes both a sweetener and a polyol, xylitol will serve only as either a sweetener or a polyol—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to oral care compositions, particularly, oral care mousse compositions, as well as oral care products thereof. The inventors discovered that certain oral care compositions and oral care products containing such oral care compositions disclosed herein can achieve a desirable mousse aesthetics. Without being limited to any particular theory, it was discovered that oral care compositions formulated to specifically have certain combinations of abrasives, surfactants systems, and thickening systems, and having certain rheological characteristics can achieve a mousse rheology and/or mousse aesthetics. It was further discovered that employing oral care packaging having certain cap and/or nozzle configurations enhances the mousiness rheology when combined with the specifically formulated oral care compositions having certain rheological characteristics. Additionally, the mousse rheology and mousse aesthetics were surprisingly achieved by embodiments disclosed herein without propellants associated the oral care product or oral care composition therein.

In accordance with an aspect of the invention, provided is an oral care product comprising: an oral care package comprising a vessel portion and a cap coupled to the vessel portion, the cap comprising a nozzle; and an oral care composition disposed in the vessel portion, the oral care composition comprising: from about 10 to about 43 wt. % of an abrasive; a surfactant system comprising: an amphoteric surfactant, and a nonionic surfactant; and a thickening system comprising: a natural gum, and an acrylic acid polymer, wherein the oral care composition has a stress drop of about 18 to about 60% and an adhesive force of about 60 to about 200 gram-force, and wherein all weight percentages are based on the total weight of the oral care composition.

According to another aspect of the invention, provided is an oral care composition comprising: from about 10 to about 43 wt. % of an abrasive; a surfactant system comprising: an amphoteric surfactant, and a nonionic surfactant; and a thickening system comprising: a natural gum, and an acrylic acid polymer, wherein the oral care composition has a stress drop of about 18 to about 60% and an adhesive force of about 60 to about 200 gram-force, and wherein all weight percentages are based on the total weight of the oral care composition.

In accordance with a further aspect of the invention, provided is an oral care composition comprising: from about 10 to about 43 wt. % of an abrasive, the abrasive comprising an alkali metal carbonate, an alkali earth metal carbonate, an insoluble phosphate, or a combination of two or more thereof; a surfactant system; a thickening system comprising: a natural gum, and an acrylic acid polymer, wherein the natural gum and the acrylic acid polymer are present in a weight ratio of the natural gum to the polyacrylic acid polymer of about 5:1 to about 1:5; from about 15 to about 44 wt. % of a polyol; and from about 20 to about 50 wt. % of water, wherein the oral care composition has a stress drop of about 18 to about 60% and an adhesive force of about 60 to about 200 gram-force, and wherein the oral care composition is free of propellants and all weight percentages are based on the total weight of the oral care composition.

In certain embodiments, the oral care composition and/or an oral care product thereof according to the present invention are substantially free or free of propellant As used herein, by “propellant or propellant material” it is meant a gaseous or volatile material, alone or in a mixture, that provides motive force to expel or aerate a composition by expansion or phase change under dispensing conditions. Propellants typically include compressed gases and liquefied gases that vaporize upon pressure release. Non-limiting examples include hydrocarbons (e.g., propane, n-butane, isobutane), halohydrocarbons (e.g., 1,1-difluoroethane, 1,1,1,2-tetrafluoroethane, hydrofluoroolefins), nitrous oxide, carbon dioxide, and dimethyl ether, whether supplied in the headspace or dissolved/dispersed in the product. Unless stated otherwise, a composition described as “free of propellants” or “propellant-free” contains no intentionally added propellant and no more than 0.01 wt. % of propellant (e.g., dissolved or incidental headspace gases), based on the total weight of the oral care composition. For example, in certain preferred embodiments, the oral care composition and/or an oral care product thereof have about 1 wt. % or less, preferably about 0.5 wt. % or less, preferably about 0.1 wt. % or less, preferably about 0.05 wt. % or less, or preferably about 0.01 wt. % or less of propellants, based on the total weight of the oral care composition. In at least one preferred embodiment, the oral care composition and/or an oral care product thereof has 0 wt. % of propellants, based on the total weight of the oral care composition.

The oral care compositions may be in the form of dentifrice, toothpaste, gels, and/or the like. Suitable components, such as those listed below, may be included or excluded from the formulations for the oral care compositions depending on the specific combination of other ingredients and the form of the oral care compositions. Additionally or alternatively, the oral care compositions may in some embodiments have a single phase, which contains that components and/or ingredients of the oral care composition. In other embodiments, the oral care composition may include two or more phases, such as two, three, four, or five phases.

The oral care compositions typically comprise an abrasive in an amount from about 10 to about 43 wt. %, based on the total weight of the oral care composition. In some embodiments, the oral care composition preferably includes a total amount of abrasive(s) of from about 10 to about 39 wt. %, about 10 to about 36 wt. %, about 10 to about 33 wt. %, about 10 to about 30 wt. %, about 10 to about 26 wt. %; from about 15 to about 43 wt. %, about 15 to about 39 wt. %, about 15 to about 36 wt. %, about 15 to about 33 wt. %, about 15 to about 30 wt. %, about 15 to about 26 wt. %; from about 19 to about 43 wt. %, about 19 to about 39 wt. %, about 19 to about 36 wt. %, about 19 to about 33 wt. %, about 19 to about 30 wt. %, about 19 to about 26 wt. %; from about 23 to about 43 wt. %, about 23 to about 39 wt. %, about 23 to about 36 wt. %, about 23 to about 33 wt. %, about 23 to about 30 wt. %; from about 26 to about 43 wt. %, about 26 to about 39 wt. %, about 26 to about 36 wt. %, about 26 to about 33 wt. %; from about 29 to about 43 wt. %, about 29 to about 39 wt. %, about 29 to about 36 wt. %, about 29 to about 34 wt. %; from about 32 to about 43 wt. %, about 32 to about 39 wt. %, about 32 to about 36 wt. %; from about 35 to about 43 wt. %, about 35 to about 39 wt. %, about 38 to about 43 wt. %, or any range or subrange thereof, based on the total weight of the oral care composition.

The abrasive(s) preferably comprise an alkali metal carbonate, an alkali earth metal carbonate, an insoluble phosphate, or a combination of two or more thereof. As used herein, alkali metal carbonates and alkali earth metal carbonates include alkali metal bicarbonates and alkali earth metal bicarbonates, respectively, unless otherwise indicated. In certain preferred embodiments, the one or more abrasives are selected from the group consisting of alkali metal carbonate, alkali earth metal carbonate, an insoluble phosphate, or a combination of two or more thereof. For example, the abrasive(s) may comprise an insoluble phosphate salts, such as calcium pyrophosphate, dicalcium orthophosphate dihydrate, tricalcium phosphate, calcium polymetaphosphate, or a combination of two or more thereof. In at least one preferred embodiment, the one or more abrasives are selected from the group consisting of insoluble phosphate salts and/or calcium-based abrasives. For instance, the one or more abrasives may be selected from the group consisting of calcium pyrophosphate, dicalcium orthophosphate dihydrate, tricalcium phosphate, calcium polymetaphosphate, calcium carbonate, calcium bicarbonate, and a combination of two or more thereof. In certain embodiments, the abrasive system may consist of one or two abrasive materials to provide the rheology and foam properties according to the present invention. In certain preferred embodiments, the calcium carbonate source is natural calcium carbonate, and free of any precipitated calcium carbonate.

The abrasive(s) may consist of alkali earth metal carbonates and alkali earth metal carbonates in certain preferred embodiments. Examples of alkali earth metal carbonates and alkali earth metal carbonates include calcium carbonate, calcium bicarbonate, potassium carbonate, potassium bicarbonate, sodium carbonate, sodium bicarbonate, zinc carbonate, zinc bicarbonate, or a combination of two or more thereof. In some cases, the alkali earth metal carbonates and alkali earth metal carbonates are chosen from calcium carbonate, calcium bicarbonate, sodium carbonate, sodium bicarbonate, potassium bicarbonate, or a combination of two or more thereof.

Preferably, the abrasive(s) comprises calcium carbonate and/or calcium bicarbonate. In some preferred embodiments, the total amount of calcium carbonate and/or calcium bicarbonate comprises about 50 wt. % or more, about 60 wt. % or more, about 70 wt. % or more, about 80 wt. % or more, about 95 wt. % or more, about 98 wt. % or more, or about 99 wt. % or more of the total amount of the abrasive agent in the oral care composition. In at least one embodiment, calcium carbonate and/or calcium bicarbonate are the sole abrasive(s) in the oral care composition.

Additional abrasives, which may be incorporated into or excluded from the oral care composition, include silica, silicate, silicon, alumina (including calcined aluminum oxide), aluminosilicates, such as bentonite, zeolite, kaolin, and mica, siliceous or diatomaceous earth, pumice, calcium compounds, calcium carbonate, cuttlebone, insoluble phosphates, composite resins, such as melamine resin, phenolic resin, and urea-formaldehyde resin, polycarbonate, silicon carbide, boron carbide, microcrystalline wax, microcrystalline cellulose, including combinations of colloidal microcrystalline cellulose and carboxymethylcellulose, and combinations and derivatives thereof.

The oral care compositions typically comprise a thickening system comprising one or more polyacrylic acid polymer, one or more polysaccharide, salt(s) thereof, or a combination of two or more thereof. In some embodiments, the thickening system consists of one or more cross-linked polyacrylic acid and/or salt(s) thereof, one or more polysaccharide and/or salt(s) thereof, and a combination of two or more thereof, such as those described herein.

The thickening system may be present in the oral care compositions in an amount ranging from about 0.5 to about 6 wt. %, based on the total weight of the oral care composition. For instance, a total amount of the thickening system may, preferably, be from about 0.5 to about 5 wt. %, about 0.5 to about 4 wt. %, about 0.5 to about 3 wt. %, about 0.5 to about 2 wt. %; from about 0.8 to about 6 wt. %, about 0.8 to about 5 wt. %, about 0.8 to about 4 wt. %, about 0.8 to about 3 wt. %, about 0.8 to about 2 wt. %; from about 1.1 to about 6 wt. %, about 1.1 to about 5 wt. %, about 1.1 to about 4 wt. %, about 1.1 to about 3 wt. %, about 1.1 to about 2 wt. %; from about 1.5 to about 6 wt. %, about 1.5 to about 5 wt. %, about 1.5 to about 4 wt. %, about 1.5 to about 3 wt. %, about 1.5 to about 2 wt. %; from about 2 to about 6 wt. %, about 2 to about 5 wt. %, about 2 to about 4 wt. %, about 2 to about 3 wt. %, about 2 to about 2 wt. %; from about 3 to about 6 wt. %, about 3 to about 5 wt. %, about 3 to about 4 wt. %; from about 4 to about 6 wt. %, about 4 to about 5 wt. %, or any range or subrange thereof, based on the total weight of the oral care composition.

The thickening system typically comprises acrylic acid polymer and/or a salt thereof, which may be present in an amount from about 0.2 to about 4 wt. %, based on the total weight of the oral care composition. In some instances, the one or more acrylic acid polymer and/or a salt thereof is, preferably, present in an amount from about 0.2 to about 3 wt. %, about 0.2 to about 2.5 wt. %, about 0.2 to about 2 wt. %, about 0.2 to about 1.5 wt. %, about 0.2 to about 1.25 wt. %, about 0.2 to about 1 wt. %, about 0.2 to about 0.75 wt. %, about 0.2 to about 0.5 wt. %; from about 0.35 to about 4 wt. %, about 0.35 to about 3 wt. %, about 0.35 to about 2.5 wt. %, about 0.35 to about 2 wt. %, about 0.35 to about 1.5 wt. %, about 0.35 to about 1.25 wt. %, about 0.35 to about 1 wt. %, about 0.35 to about 0.75 wt. %, about 0.35 to about 0.5 wt. %; from about 0.5 to about 4 wt. %, about 0.5 to about 3 wt. %, about 0.5 to about 2.5 wt. %, about 0.5 to about 2 wt. %, about 0.5 to about 1.5 wt. %, about 0.5 to about 1.25 wt. %, about 0.5 to about 1 wt. %, about 0.5 to about 0.75 wt. %; from about 0.75 to about 4 wt. %, about 0.75 to about 3 wt. %, about 0.75 to about 2.5 wt. %, about 0.75 to about 2 wt. %, about 0.75 to about 1.5 wt. %, about 0.75 to about 1.25 wt. %, about 0.75 to about 1 wt. %; from about 1 to about 4 wt. %, about 1 to about 3 wt. %, about 1 to about 2.5 wt. %, about 1 to about 2 wt. %, about 1 to about 1.5 wt. %, about 1 to about 1.25 wt. %; from about 1.5 to about 4 wt. %, about 1.5 to about 3 wt. %, about 1.5 to about 2.5 wt. %, about 1.5 to about 2 wt. %; from about 2 to about 4 wt. %, about 2 to about 3 wt. %, about 2 to about 2.5 wt. %; from about 2.5 to about 4 wt. %, about 2.5 to about 3 wt. %, about 3 to about 4 wt. %, or any range or subrange thereof, based on the total weight of the oral care composition.

The acrylic acid polymer and/or a salt thereof may be chosen from acrylates, cross-linked polyacrylic acids, and/or salt(s) thereof. In some preferred embodiments, the acrylic acid polymer comprises a cross-linked polyacrylic acid and/or a salt thereof. The cross-linked polyacrylic acid and/or a salt thereof may be a branched polyacrylic acid and/or salt thereof. In some instances, the polyacrylic acid and/or a salt thereof consists of branched polyacrylic acids and/or salts thereof. In further instances, however, the oral care composition includes at least one linear polyacrylic acid and/or a salt thereof.

The cross-linked polyacrylic acid and/or a salt thereof may be a carbomer. The carbomer may comprise or be selected from the group consisting of Carbopols. For instance, the personal care composition may include, or in some cases exclude, a polyacrylic acid and/or a salt thereof selected from Carbopol 2984, Carbopol 910, Carbopol 934P, Carbopol Ultrez 10, Carbopol 940, Carbopol 941, Carbopol 956, Carbopol 971P, Carbopol 974P, Carbopol 981, Carbopol 2020, and a combination of two or more thereof. In some cases, the polyacrylic acid and/or a salt thereof comprises one or more of or is selected from the group consisting of Carbopol 2984, Carbopol 910, Carbopol 934P, Carbopol Ultrez 10, Carbopol 940, Carbopol 941, Carbopol 956, Carbopol 971P, Carbopol 974P, Carbopol 981, Carbopol 2020, and/or a combination of two or more thereof. In at least one embodiment, the carbomer comprises or consists of Carbopol ETD NF 2020. Additionally or alternatively, the cross-linked polyacrylic acid and/or a salt thereof may consists solely of carbomers.

The polyacrylic acid polymer may comprise an acrylate. Examples of acrylates that may be included in the oral care composition include, e.g., isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl acrylate, 2-phenoxyethyl acrylate, tetrahydrofurfuryl acrylate, 2-hydroxyethyl acrylate, 2-hydroxypropyl acrylate, 4-hydroxybutyl acrylate, dimethylaminoethyl acrylate, 1,4-butanediol acrylate, or a combination of two or more thereof.

The acrylate may be chosen from diacrylates. In some embodiments, the oral care composition includes a diacrylate chosen from 1,4-butanediol, 1,6-hexanediol, tetraethylene glycol, tripropylene glycol, ethoxylated bisphenol-A, and a combination of two or more thereof. Triacrylate monomers include trimethylol propane, ethoxylated, glyceryl propoxy, and pentaerythritol.

Acrylates further include methacrylates, such as methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylopropane methacrylate, ethoxyethyl methacrylate, trifluoroethyl methacrylate, or a combination of two or more thereof. In some embodiments, the thickening system is substantially free of or free of methacrylates and/or diacrylates.

The one or more thickening agent may be selected from a polysaccharide, e.g., in an amount from about 0.01 wt. % to about 4 wt. %, based on the total weight of the oral care composition. In some instances, the one or more polysaccharide may, preferably, be present in an amount from about 0.02 to about 4 wt. %, about 0.03 to about 3.5 wt. %, about 0.03 to about 3 wt. %, about 0.02 to about 1.5 wt. %, about 0.02 to about 1.25 wt. %, about 0.02 to about 1 wt. %, about 0.02 to about 0.75 wt. %, about 0.02 to about 0.5 wt. %; from about 0.03 to about 4 wt. %, about 0.03 to about 3 wt. %, about 0.035 to about 2.5 wt. %, about 0.035 to about 2 wt. %, about 0.35 to about 1.5 wt. %, about 0.35 to about 1.25 wt. %, about 0.35 to about 1 wt. %, about 0.35 to about 0.75 wt. %, about 0.35 to about 0.5 wt. %; from about 0.5 to about 4 wt. %, about 0.5 to about 3 wt. %, about 0.5 to about 2.5 wt. %, about 0.5 to about 2 wt. %, about 0.5 to about 1.5 wt. %, about 0.5 to about 1.25 wt. %, about 0.5 to about 1 wt. %, about 0.5 to about 0.75 wt. %; from about 0.75 to about 4 wt. %, about 0.75 to about 3 wt. %, about 0.75 to about 2.5 wt. %, about 0.75 to about 2 wt. %, about 0.75 to about 1.5 wt. %, about 0.75 to about 1.25 wt. %, about 0.75 to about 1 wt. %; from about 1 to about 4 wt. %, about 1 to about 3 wt. %, about 1 to about 2.5 wt. %, about 1 to about 2 wt. %, about 1 to about 1.5 wt. %, about 1 to about 1.25 wt. %; from about 1.5 to about 4 wt. %, about 1.5 to about 3 wt. %, about 1.5 to about 2.5 wt. %, about 1.5 to about 2 wt. %; from about 2 to about 4 wt. %, about 2 to about 3 wt. %, about 2 to about 2.5 wt. %; from about 2.5 to about 4 wt. %, about 2.5 to about 3 wt. %, about 3 to about 4 wt. %, or any range or subrange thereof, based on the total weight of the oral care composition.

The polysaccharide may be selected from celluloses, cellulose derivatives, natural gum, and combinations thereof. In some embodiments, the polysaccharide is a natural gum. Examples of natural gums include alginate, pectin, carrageenan, gum arabic, tragacanth gum, karaya gum, agarose, guar gum, pectin, gellan gum, tara gum, locust bean gum, tamarind gum, locust bean gum, Seneca gum, sclerotium gum, gellan gum, xanthan gum, or a combination of two or more thereof. For instance, the thickening system may include a natural gum comprising gum arabic, tragacanth gum, karaya gum, agarose, guar gum, pectin, gellan gum, tara gum, locust bean gum, tamarind gum, locust bean gum, Seneca gum, sclerotium gum, gellan gum, xanthan gum, or a combination thereof. In some cases, the oral care composition includes a natural gum selected from the group consisting of xanthan gum, carrageenan, pectin, alginate, pectin, and a combination of two or more thereof. In at least one embodiment, the natural gum comprises xanthan gum. In another embodiment, the natural gum consists solely of xanthan gum.

In some embodiments, the xanthan gum is present in an amount of from about 0.01 wt. % to about 0.5 wt. %, or from about 0.02 wt. % to about 0.5 wt. %, or from about 0.03 wt. % to about 0.5 wt. %, or from about 0.04 wt. % to about 0.4 wt. %, or about 0.04 wt. %, or about 0.3 wt. %, based on the total weight of the composition. In some embodiments, the xanthan gum is present in an amount of from 0.01 wt. % to 0.5 wt. %, or from 0.02 wt. % to 0.5 wt. %, or from 0.03 wt. % to 0.5 wt. %, or from 0.04 wt. % to 0.4 wt. %, or 0.04 wt. %, or 0.3 wt. %, based on the total weight of the composition.

The polysaccharide may be a cellulose. Examples of celluloses include cellulose ether chosen from carboxymethylcellulose (CMC), ethylcellulose (EC), hydroxypropylcellulose (HPC), hydroxypropylmethylcellulose (HPMC), methylhydroxyethylcellulose (MHEC), and mixtures thereof. In some embodiments, the cellulose is a modified cellulose selected from microcrystalline cellulose (MCC), e.g., in the anhydrous or hydrated forms thereof, and cellulose ethers such as hydroxypropylcellulose (HPC) and hydroxypropylmethylcellulose (HPMC). In at least one embodiment, the oral care composition has a thickening system including one or more cellulose selected from the group consisting of carboxymethylcellulose, ethylcellulose, hydroxypropylcellulose, hydroxypropylmethylcellulose, methylhydroxyethylcellulose, and a combination thereof. In at least one preferred embodiment, the thickening system includes a polysaccharide including one or more cellulose comprising or consisting ofcarboxymethylcellulose.

Preferably, the thickening system is adapted to have a weight ratio of the total amount of natural gum (preferably, xanthan gum) to the total amount of polyacrylic acid polymer of about 5:1 to about 1:5. In some preferred embodiments, the weight ratio of the total amount of natural gum (preferably, xanthan gum) to the total amount of polyacrylic acid polymer of about 5:1 to about 1:5, about 4:1 to about 1:5, about 3:1 to about 1:5, about 2:1 to about 1:5, about 1:1 to about 1:5; from about 5:1 to about 1:4, about 4:1 to about 1:4, about 3:1 to about 1:4, about 2:1 to about 1:4, about 1:1 to about 1:4; from about 5:1 to about 1:3, about 4:1 to about 1:3, about 3:1 to about 1:3, about 2:1 to about 1:5, about 1:1 to about 1:3; from about 5:1 to about 1:2, about 4:1 to about 1:2, about 3:1 to about 1:2, about 2:1 to about 1:2, about 1:1 to about 1:2; from about 5:1 to about 1:1, about 4:1 to about 1:1, about 3:1 to about 1:1, about 2:1 to about 1:1, or any range or subrange thereof.

The oral care compositions typically include a surfactant system comprising a plurality of surfactants. The surfactant system may be present in the oral care composition in an amount from about 0.5 to about 9 wt. %, based on the total weight of the oral care composition. For example, the oral care composition may include a surfactant system in an amount from about 0.5 to about 8 wt. %, about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 9 wt. %, about 1 to about 8 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 2 to about 9 wt. %, about 2 to about 8 wt. %, about 2 to about 7 wt. %, about 2 to about 6 wt. %, about 2 to about 5 wt. %, about 2 to about 4 wt. %, about 2 to about 3 wt. %; from about 2.5 to about 9 wt. %, about 2.5 to about 8 wt. %, about 2.5 to about 7 wt. %, about 2.5 to about 6 wt. %, about 2.5 to about 5 wt. %, about 2.5 to about 4 wt. %, about 2.5 to about 3.5 wt. %; from about 3 to about 9 wt. %, about 3 to about 8 wt. %, about 3 to about 7 wt. %, about 3 to about 6 wt. %, about 3 to about 5 wt. %, about 3 to about 4 wt. %, about 3 to about 3.5 wt. %; from about 4 to about 9 wt. %, about 4 to about 8 wt. %, about 4 to about 7 wt. %, about 4 to about 6 wt. %, about 4 to about 5 wt. %; from about 5 to about 9 wt. %, about 5 to about 8 wt. %, about 5 to about 7 wt. %, about 5 to about 6 wt. %; from about 7 to about 9 wt. %, about 7 to about 8 wt. %, or any range or subrange thereof, based on the total weight of the oral care composition.

The surfactant system may comprise or consist of one or more anionic surfactant, one or more amphoteric surfactants, one or more nonionic surfactants, and/or combinations of two or more thereof. For example, the surfactant system may comprise one or more non-sulfate based anionic surfactant, one or more amphoteric surfactant, and one or more nonionic surfactant.

The oral care compositions typically comprise a surfactant system having one or more anionic surfactant(s). The surfactant system may include one or more anionic surfactant(s) in an amount that may range from about 0.1 to about 5 wt. %, based on the total weight of the oral care composition. In some instances, the one or more anionic surfactant(s) is present in the surfactant system in an amount from about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1.5 wt. %; from about 1 to about 5 wt. %, about 1 to about 4 wt. %, about 1 to about 3 wt. %, about 1 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %, about 3 to about 5 wt. %, or any range or subrange thereof, based on the total weight of the oral care composition.

The one or more anionic surfactant(s) may be selected from non-sulfate based anionic surfactants, such as sulfonated monoglycerides of fatty acid surfactants, isethionate surfactants, sarcosinate surfactants, taurate surfactants, and a combination of two or more thereof. Although the one or more anionic surfactant(s) may be chosen from non-sulfate based anionic surfactants, the oral care composition may, in some embodiments, include a sulfate based anionic surfactants selected from sodium lauryl sulfate, sodium lauryl ether sulfate, ammonium lauryl sulfate, ammonium lauryl ether sulfate, sodium dodecyl sulfate, sodium coco-sulfate, ammonium coco-sulfate, and a combination of two or more thereof. In at least one embodiment, the surfactant system includes a sulfate based anionic surfactant, wherein the oral care composition is substantially free of or free of sodium lauryl sulfate and/or sodium lauryl ether sulfate.

The anionic surfactant(s) may have at least one acyl group, preferably, comprising a carbon chain of 8 to 21 carbons. In some instance, the alkyl group(s) of the anionic surfactant(s) comprise a carbon chain of 8 to 19 carbons, 8 to 17 carbons, 8 to 15 carbons, 8 to 13 carbons, 8 to 11 carbons; 9 to 21 carbons, 9 to 19 carbons, 9 to 17 carbons, 9 to 15 carbons, 9 to 13 carbons, 9 to 11 carbons; 11 to 21 carbons, 11 to 19 carbons, 11 to 17 carbons, 11 to 15 carbons, 11 to 13 carbons; 13 to 21 carbons, 13 to 19 carbons, 13 to 17 carbons, or any range or subrange thereof. The anionic surfactants disclosed herein may be incorporated in a salt form. The salt form of the anionic surfactants may have an alkali metal (e.g., sodium or potassium) and/or ammonium group.

Non-limiting examples of isethionates include sodium isethionate, sodium cocoyl isethionate, sodium lauroyl methyl isethionate, and sodium cocoyl methyl isethionate. Sulfonated monoglycerides of fatty acids include sodium coconut monoglyceride sulfonates and the like. Examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations of two or more thereof.

The anionic surfactant may be selected from taurates having a structure according to the following formula:

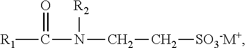

wherein R₁ is a saturated or unsaturated, straight or branched alkyl chain with 6 to 18 carbon atoms, R₂ is H or methyl, and M⁺ is H, sodium, or potassium (e.g., sodium methyl cocoyl taurate).

Additional examples of taurate surfactants include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations of two or more thereof. In some embodiments, the oral care composition comprises sodium lauroyl methyl taurate (or sodium methyl lauroyl taurate), sodium methyl cocoyl taurate (SMCT), or a combination thereof. In at least one preferred embodiment, the surfactant system comprises sodium methyl cocoyl taurate.

The surfactant system may comprise one or more amphoteric surfactant(s), e.g., in an amount from about 0.1 to about 5 wt. %, based on the total weight of the oral care composition. For example, the one or more amphoteric surfactant(s) may be present in the surfactant system in an amount from about 0.1 to about 4 wt. %, about 0.1 to about 3 wt. %, about 0.1 to about 2 wt. %, about 0.1 to about 1 wt. %; from about 0.3 to about 5 wt. %, about 0.3 to about 4 wt. %, about 0.3 to about 3 wt. %, about 0.3 to about 2 wt. %, about 0.3 to about 1 wt. %; from about 0.6 to about 5 wt. %, about 0.6 to about 4 wt. %, about 0.6 to about 3 wt. %, about 0.6 to about 2 wt. %, about 0.6 to about 1 wt. %; from about 1 to about 5 wt. %, about 1 to about 4 wt. %, about 1 to about 3 wt. %, about 1 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, including any range or subrange thereof, based on the total weight of the oral care composition.

Amphoteric surfactants are typically characterized by a combination of high surfactant activity, lather forming and mildness. The amphoteric surfactant may comprise a substituent containing 8 to 18 carbon atoms and a substituent containing one or more carboxylate, sulfonate, sulfate, phosphate, or phosphonate. For instance, the amphoteric surfactant may have an alkyl group comprising from 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched. In some cases, one of the aliphatic substituents of the amphoteric surfactant contains about 8 to about 18 carbon atoms and one of the aliphatic substituents contains an anionic water solubilizing group, e.g., carboxy, sulfonate, sulfate, phosphate, or phosphonate. The amphoteric surfactants disclosed herein may be present in the oral care composition in a salt form.

The amphoteric surfactants may include alkyl amphopropionate surfactants, betaine surfactants, alkyl sultaine surfactants, alkyl amphoacetate surfactants, or a combination of two or more thereof. Preferably, the oral care composition includes an amphoteric surfactant selected from betaine surfactants (also referred to herein as betaines). Examples of betaine surfactants include, e.g., alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl) carboxy methyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine. In some instances, the betaine surfactant is selected from coca betaine, cocamidopropyl betaine, lauryl betaine, laurylhydroxy sulfobetaine, lauryldimethyl betaine, behenyl betaine, capryl/capramidopropyl betaine, lauryl hydroxysultaine, stearyl betaine, and a combination of two or more thereof. For instance, the betaine surfactant may be coco betaine, cocamidopropyl betaine, behenyl betaine, capryl/capramidopropyl betaine, and lauryl betaine, or a combination of two or more thereof. In at least one embodiment, the oral care composition comprises or consists of coco betaine, cocamidopropyl betaine, or a combination thereof.

The surfactant system may comprise one or more nonionic surfactant(s). The one or more nonionic surfactant(s) may be present in an amount from about 0.1 to about 7 wt. %, based on the total weight of the oral care composition. In some instances, the one or more nonionic surfactant(s) is present in the surfactant system in an amount from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 7 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 7 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.4 to about 7 wt. %, about 1.4 to about 6 wt. %, about 1.4 to about 5 wt. %, about 1.4 to about 4 wt. %, about 1.4 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 5 to about 7 wt. %, or any range or subrange thereof, based on the total weight of the oral care composition.

The one or more nonionic surfactant(s) may be selected from glycosides and/or glucosides compounds produced by the condensation of alkylene oxide groups (hydrophilic in nature) with an organic hydrophobic compound, which may be aliphatic or alkylaromatic in nature. The non-ionic surfactant may comprise or consist of glycosides, such as C₁₀₋₁₆-alkyl glycosides, C₈₋₂₀-alkyl glycosides, C₁₀₋₂₀-alkyl glycosides, C₁₂₋₂₀-alkyl glycosides, C₈₋₁₈-alkyl glycosides, C₈₋₁₆-alkyl glycosides, C₈₋₁₄-alkyl glycosides, C₁₀₋₁₈-alkyl glycosides, and/or C₁₀₋₁₆-alkyl glycosides. In at least one preferred embodiment, the nonionic surfactants comprise or consist solely of C₁₀₋₁₆-alkyl glycoside, such as D-glucopyranose, oligomeric, C₁₀₋₁₆-alkyl glycosides.

The glycosides may be selected from the group consisting of glucosides. Examples of glucoside surfactants include decyl glucoside, stearyl glucoside, lauryl glucoside, coco-glucoside, cetearyl glucoside, decyl lauryl glucoside, lauryl ethyl glucoside, myristoyl ethyl glucoside, oleoyl ethyl glucoside, or a combination of two or more thereof. In some embodiments, the surfactant system includes one or more nonionic surfactant(s) chosen from lauryl glucoside, lauryl ethyl glucoside, myristoyl ethyl glucoside, oleoyl ethyl glucoside, and a combination of two or more thereof. Additionally or alternatively, the glucoside surfactant may be chosen from polyglucosides, such as alkylpolyglucosides.

Further examples of nonionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials. Additional examples of nonionic surfactants include polyoxyethylene, polyoxyethylene sorbitan esters, polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, or a combination of two or more thereof. In some instances, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, or a combination of two or more thereof.

The oral care composition may be substantially free of or free of cationic surfactants. For example, the oral care composition and/or an oral care product thereof may have about 2 wt. % or less, about 1 wt. % or less, about 0.5 wt. % or less, about 0.1 wt. % or less, about 0.05 wt. % or less of cationic surfactants, based on the total weight of the oral care composition. In at least one preferred embodiment, the oral care composition and/or an oral care product thereof has 0 wt. % of cationic surfactants, based on the total weight of the oral care composition.

The oral care compositions may include one or more polyol(s). In some cases, the oral care composition includes a polyol system comprising a plurality of polyols. The total amount of polyols present in the oral care composition about 15 to about 44 wt. %, about 15 to about 39 wt. %, about 15 to about 35 wt. %, about 15 to about 31 wt. %, about 15 to about 28 wt. %, about 15 to about 25 wt. %; from about 19 to about 44 wt. %, about 19 to about 39 wt. %, about 19 to about 35 wt. %, about 19 to about 31 wt. %, about 19 to about 28 wt. %, about 19 to about 25 wt. %; from about 23 to about 44 wt. %, about 23 to about 39 wt. %, about 23 to about 35 wt. %, about 23 to about 31 wt. %; from about 27 to about 44 wt. %, about 27 to about 39 wt. %, about 27 to about 35 wt. %, or any range or subrange thereof, based on the total weight of the oral care composition.

The polyol(s) may be chosen from glycols or compounds with numerous hydroxyl groups. The one or more polyols may be liquid at ambient temperature (25° C.). The one or more polyols may have from 2 to 32 carbon atoms, from 3 to 16 carbon atoms, or from 3 to 12 carbon atoms. In some preferred embodiments, the polyol(s) comprises glycerin, glycol, inositol, maltitol, mannitol, sorbitol, xylitol, propylene glycol, polypropylene glycol (PPG), polyethylene glycol (PEG), a saccharide (e.g., fructose, glucose, sucrose and mixtures of saccharides, such as honey), or a combination of two or more thereof. For instance, the oral care composition may comprise glycerin, inositol, maltitol, mannitol, sorbitol, xylitol, propylene glycol, polypropylene glycol, polyethylene glycol, or a combination of two or more thereof.

The polyol may be a humectant including, e.g., glycol, sorbitol, glycerin, inositol, maltitol, mannitol, xylitol, propylene glycol, polypropylene glycol (PPG), polyethylene glycol (PEG), a saccharide, polyoxypropylene-polyoxyethylene block copolymer, and a combination of two or more thereof. Examples of glycols include ethylene glycol, propylene glycol, butylene glycol, hexylene glycol, diethylene glycol, dipropylene glycol, polypropylene glycol (PPG), polyethylene glycol (PEG), a copolymer of polypropylene glycol (PPG), polyethylene glycol (PEG), or a combination of two or more thereof.

Additional, non-limiting examples of polyols that may, optionally, be included in the oral care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture of two or more thereof.

Additionally or alternatively, the oral care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof. In some embodiments, the polyol is a polyethylene glycol, such as polyethylene glycol 600. In some embodiments, the oral care composition comprises one or more polyethylene glycols, e.g., polyethylene glycols in a molecular weight range from 200 to 800. For example, the compositions may comprise one or more of polyethylene glycol 200, polyethylene glycol 300, polyethylene glycol 400, polyethylene glycol, 600 or polyethylene glycol 800.

The oral care composition may include water in an amount from about 20 to about 50 wt. %, based on the total weight of the oral care composition. Preferably, the total amount of water in the oral care composition is from about 20 to about 46 wt. %, about 20 to about 42 wt. %, about 20 to about 40 wt. %, about 20 to about 38 wt. %, about 20 to about 35 wt. %; from about 24 to about 50 wt. %, about 24 to about 46 wt. %, about 24 to about 42 wt. %, about 24 to about 40 wt. %, about 24 to about 38 wt. %, about 24 to about 35 wt. %; from about 28 to about 50 wt. %, about 28 to about 46 wt. %, about 28 to about 42 wt. %, about 28 to about 40 wt. %, about 28 to about 38 wt. %, about 28 to about 35 wt. %; from about 30 to about 50 wt. %, about 30 to about 46 wt. %, about 30 to about 42 wt. %, about 30 to about 40 wt. %; from about 32 to about 50 wt. %, about 32 to about 46 wt. %, about 32 to about 42 wt. %; from about 34 to about 50 wt. %, about 34 to about 46 wt. %, about 34 to about 42 wt. %; from about 36 to about 50 wt. %, about 36 to about 46 wt. %, about 36 to about 42 wt. %, or any range or subrange thereof, based on the total weight of the oral care composition.

The oral care compositions may include a fluoride ion source. The fluoride ion source may be present in an effective amount. In some cases, the fluoride ion source is present in the oral care composition in an amount of about 0.01 to about 5 wt. %, based on the total weight of the oral care composition. For example, the fluoride ion source is present in an amount of about 0.01 to about 5 wt. %, about 0.01 to about 4 wt. %, about 0.01 to about 3 wt. %, about 0.01 to about 2 wt. %, about 0.01 to about 1 wt. %; from about 0.05 to about 5 wt. %, about 0.05 to about 4 wt. %, about 0.05 to about 3 wt. %, about 0.05 to about 2 wt. %, about 0.05 to about 1 wt. %; from about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 5 wt. %, about 1.2 to about 4 wt. %, about 1.2 to about 3 wt. %, about 1.2 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 4 to about 5 wt. %, or any range or subrange thereof, based on the total weight of the oral care composition.

The fluoride ion source may be selected from soluble fluoride ion salts. For example, the fluoride ion source may comprise sodium fluoride, potassium fluoride, calcium fluoride, zinc fluoride, zinc ammonium fluoride, lithium fluoride, ammonium fluoride, stannous fluoride, stannous fluorozirconate, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, amine fluorides or a combination of two or more thereof. In some embodiments, the fluoride ion source comprises sodium fluoride, sodium monofluorophosphate, or a combination thereof. Additional examples of fluoride ion source are disclosed in U.S. Pat. Nos. 3,535,421, 4,885,155, and 3,678,154, the disclosure of each of which is hereby incorporated by reference in their entirety.

In certain embodiments, the oral care composition of the disclosure may contain stannous fluoride and a source of fluoride ions or fluorine-providing agents in amounts sufficient to deliver, in total, from 25 ppm to 25,000 ppm (mass fraction) of fluoride ions, generally at least 500 ppm, e.g., from about 500 to about 2000 ppm, from about 800 to about 1800 ppm, from about 1000 to about 1600 ppm, from about 1200 to about 1550 ppm, or about 1450 ppm.

The oral care compositions may include an anti-calculus agent. The amount of anti-calculus agent(s) present in the oral are composition may be from about 0.5 to about 10 wt. %, based on the total weight of the oral care composition. In some instances, the anti-calculus agent(s) is present in the oral care composition in an amount from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 5 to about 10 wt. %, about 5 to about 8 wt. %, about 5 to about 6 wt. %; from about 7 to about 10 wt. %, about 7 to about 8 wt. %, about 8 to about 10 wt. %, or any range or subrange thereof, based on the total weight of the oral care composition.

The anti-calculus agent(s) may be chosen from phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and combinations of two or more thereof. Examples of anti-calculus agents include tetrasodium pyrophosphate, ammonium tripolyphosphate, hexametaphosphate salt, polycarboxylate polymers, polyvinyl methyl ether/maleic anhydride (PVME/MA) copolymers, or combinations of two or more thereof. In some embodiments, the oral care composition includes one or more alkali metal polyphosphate, e.g., as tetrasodium pyrophosphate.

The oral care compositions of the present disclosure may include a flavoring agent. The flavoring agent is typically incorporated in the oral care composition at a concentration of about 0.01 to about 3 wt. % by weight of the oral care composition. For example, the amount of flavoring agent(s) present in the oral care composition may be from about 0.01 to about 2 wt. %, about 0.01 to about 1 wt. %, about 0.01 to about 0.5 wt. %, about 0.01 to about 0.1 wt. %; from about 0.1 to about 3 wt. %, about 0.1 to about 2 wt. %, about 0.1 to about 1 wt. %, about 0.1 to about 0.5 wt. %; from about 0.5 to about 3 wt. %, about 0.5 to about 2 wt. %, about 0.5 to about 1 wt. %; from about 1 to about 3 wt. %, about 1 to about 2 wt. %; from about 2 to about 3 wt. %, including any range or subrange thereof, based on the total weight of the oral care composition.

Suitable flavoring agents include, but are not limited to, essential oils and various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Additional flavoring agents may include, but are not limited to menthol, artificial vanilla, cinnamon derivatives, and various fruit flavors, spearmint oil, peppermint oil, cinnamon oil, oil of wintergreen (methylsalicylate), clove oil, bay oil, anise oil, eucalyptus oil, thyme oil, cedar leaf oil, oil of nutmeg, oil of sage, oil of bitter almonds, cassia oil, and a combination of two or more thereof.

In some embodiments, the oral care compositions may include one or more sweeteners. The oral care composition may include caloric sweeteners and/or non-caloric sweeteners. Examples of non-caloric sweeteners include saccharin, for example, sodium saccharin, acesulfame, neotame, cyclamate, sucralose, erythritol; natural high-intensity sweeteners, such as thaumatin, stevioside, rebaudiosides (e.g., rebaudioside A), or glycyrrhizin; or sugar alcohols, such as sorbitol, xylitol, maltitol and mannitol. Examples of caloric sweeteners include sugars, such as fructose, glucose, sucrose, and high fructose syrups. In at least one embodiment, the one or more sweeteners are chosen from rebaudiosides (preferably, rebaudioside A), xylitol, erythritol, and a combination of two or more thereof.

The one or more sweetener(s) may be present in the oral care composition in an amount from about 0.1 to about 15 wt. %, based on the total weight of the oral care composition. For example, the oral care composition may have a total amount of sweetener(s) from about 0.1 to about 12 wt. %, about 0.1 to about 10 wt. %, about 0.1 to about 5 wt. %, about 0.1 to about 3 wt. %, about 0.1 to about 1 wt. %; from about 0.3 to about 12 wt. %, about 0.3 to about 10 wt. %, about 0.3 to about 5 wt. %, about 0.3 to about 3 wt. %, about 0.3 to about 1 wt. %; from about 1 to about 12 wt. %, about 1 to about 10 wt. %, about 1 to about 5 wt. %, about 1 to about 3 wt. %; from about 2 to about 12 wt. %, about 2 to about 10 wt. %, about 2 to about 5 wt. %; from about 4 to about 12 wt. %, about 4 to about 10 wt. %, about 4 to about 7 wt. %; from about 7 to about 12 wt. %, about 7 to about 11 wt. %, about 9 to about 12 wt. %, about 9 to about 11 wt. %, or any range or subrange thereof, based on the total weight of the oral care composition.

In some embodiments, the oral care composition preferably is substantially free or free of caloric sweeteners. For example, the oral care compositions may have about 4 wt. % or less, about 3 wt. % or less, about 2 wt. % or less, about 1 wt. % or less, about 0.5 wt. % or less, or about 0.1 wt. % or less of caloric sweeteners, based on the weight of the oral care composition. In at least one embodiment, the oral care composition contains about 0 wt. % or 0 wt. % of caloric sweeteners, based on the weight of the oral care composition.

The oral care compositions may include one or more colorants. Exemplary colorants can include natural or uncertified colors from natural sources or certified colors for the effect of color. In some embodiments, the colorant can include dyes, certified aluminum lakes or colors derived from a natural source. The colorant may be water-based, oil-based or dry. The colorants can be primary colors, blends of colors or discrete mixtures of colors, such as confetti. The concentrations of the colorant in the oral care composition may be from trace amount to about 0.6 wt. %, from about 0.1 to about 0.5 wt. %, about 0.2 to about 0.4 wt. %, or about 0.15 to about 0.35 wt. %, based on the total weight of the oral care composition.

The oral care composition may include one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the oral care composition.

The oral care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween.

As mentioned above, it was discovered that oral care compositions formulated to specifically have certain abrasives, surfactants systems, and thickening systems, and having certain rheological characteristics can achieve a mousse rheology. Moreover, such oral care compositions when combined with certain nozzles for the oral care product achieve an enhanced mousse rheology and/or mousse aesthetics. The mousse rheology and mousse aesthetics can surprisingly be achieved without propellants associated the oral care product or oral care composition therein.

The oral care compositions are formulated to specifically have both a stress drop of about 18 to about 60% and an adhesive force of about 60 to about 200 gram-force. For instance, the preferred oral care compositions have a stress drop that is from about 18 to about 60%, measured using a DHR rheometer having a four paddled vane geometry to apply a constant shear rate of 0.05/sec on the paste for 400 seconds. In some embodiments, the stress drop of the oral care composition is from about 18 to about 60%, about 18 to about 54%, about 18 to about 48%, about 18 to about 44%, about 18 to about 40%, about 18 to about 37%, about 18 to about 34%, about 18 to about 31%, about 18 to about 28%, about 18 to about 26%; from about 21 to about 60%, about 21 to about 54%, about 21 to about 48%, about 21 to about 44%, about 21 to about 40%, about 21 to about 37%, about 21 to about 34%, about 21 to about 31%, about 21 to about 28%, about 21 to about 26%; from about 24 to about 60%, about 24 to about 54%, about 24 to about 48%, about 24 to about 44%, about 24 to about 40%, about 24 to about 37%, about 24 to about 34%; from about 27 to about 60%, about 27 to about 54%, about 27 to about 48%, about 27 to about 44%, about 27 to about 40%, about 27 to about 37%, about 27 to about 34%; from about 31 to about 60%, about 31 to about 54%, about 31 to about 48%, about 31 to about 44%, about 31 to about 40%, about 31 to about 37%; from about 36 to about 60%, about 36 to about 54%, about 36 to about 48%, about 36 to about 44%, about 36 to about 40%; from about 41 to about 60%, about 41 to about 54%, about 41 to about 48%; from about 46 to about 60%, about 46 to about 54%, about 51 to about 60%, including any range or subrange thereof, e.g., measured using a DHR rheometer having a four paddled vane geometry to apply a constant shear rate of 0.05/sec on the paste for 400 seconds.

In addition to the foregoing stress drop, the oral care compositions preferably have an adhesive force of about 60 to about 200 gram-force, measured as the peak adhesive force using a texture analyzer by applying 50 grams of compressive force on the oral care composition for 20 seconds and determining the peak adhesive force as texture analyzer withdraws after applying the compression force. In certain preferred embodiments, the oral care compositions have an adhesive force of about 60 to about 200 gram-force, about 60 to about 160 gram-force, about 60 to about 140 gram-force, about 60 to about 120 gram-force, about 60 to about 100 gram-force, about 60 to about 90 gram-force; from about 80 to about 200 gram-force, about 80 to about 160 gram-force, about 80 to about 140 gram-force, about 80 to about 120 gram-force, about 80 to about 100 gram-force; from about 100 to about 200 gram-force, about 100 to about 160 gram-force, about 100 to about 140 gram-force, about 100 to about 120 gram-force; from about 130 to about 200 gram-force, about 130 to about 160 gram-force, about 130 to about 150 gram-force; from about 160 to about 200 gram-force, about 160 to about 180 gram-force, including any range or subrange thereof, e.g., measured as the peak adhesive force using a texture analyzer by applying 50 grams of compressive force on the oral care composition for 20 seconds and determining the peak adhesive force as texture analyzer withdraws after applying the compression force.

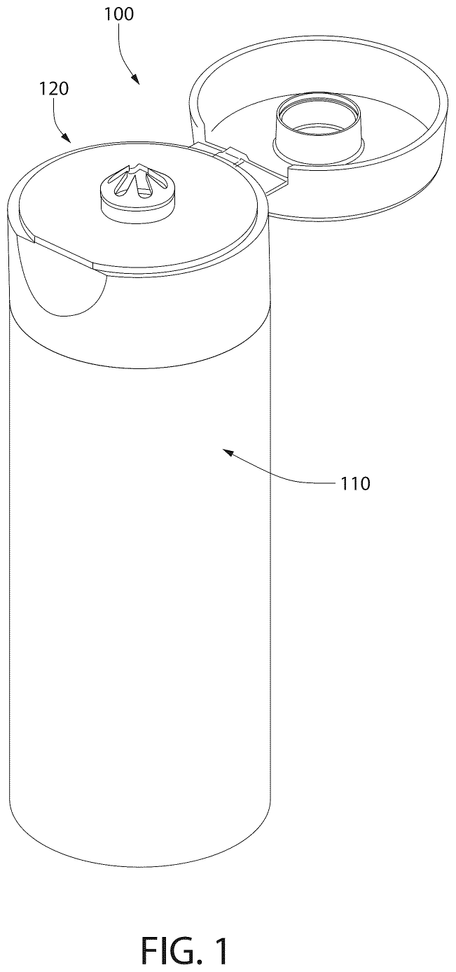

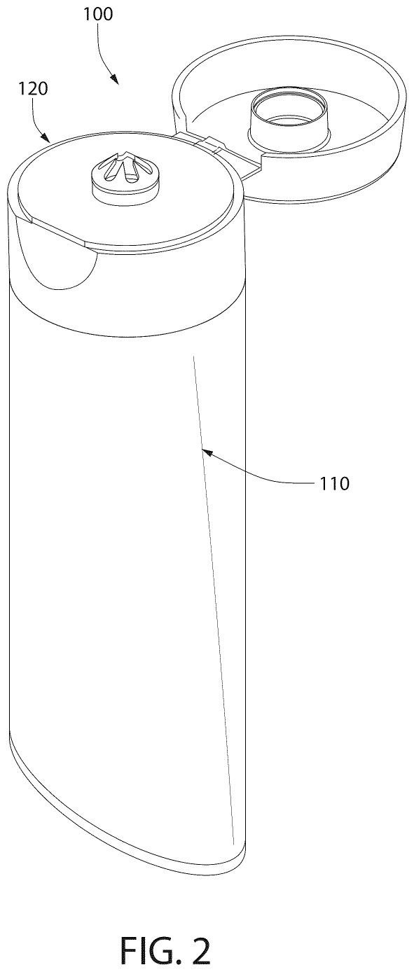

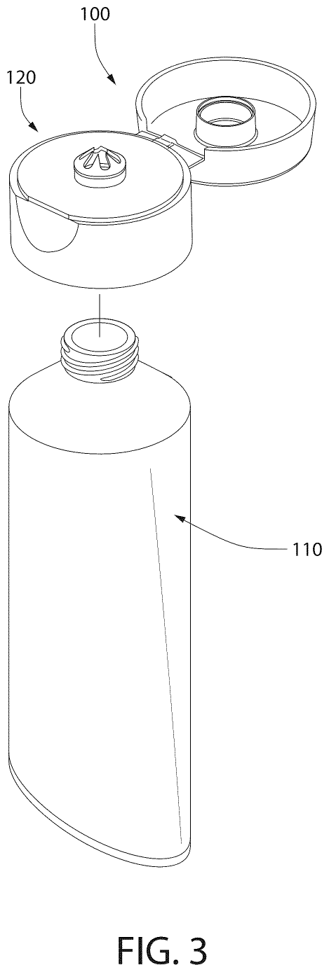

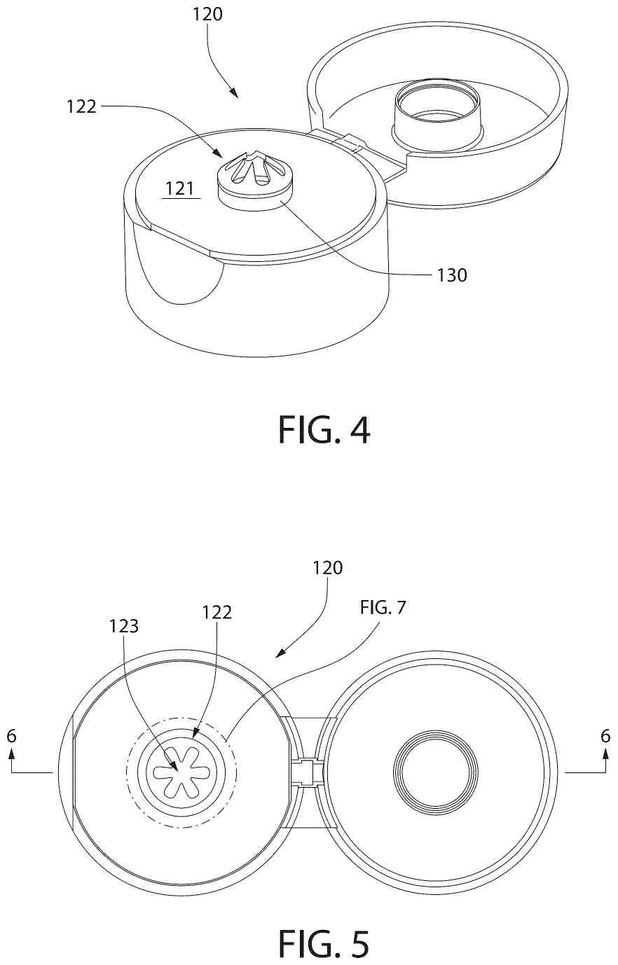

In certain embodiments, the compositions according to the present invention is provides superior and unique bubble size distribution as measured by the diameter of the bubbles to provide a continuous foam phase as measured by known imaging methodologies and counting statistics in the bulk foam formed or dispensed nurdle. In certain embodiments, the median bubble size (50% of the formed bubble, D50) of the bubbles may be smaller than 95, 90, 75 or 50 μm. In certain embodiments, the diameter of 90% of the bubbles (D90), are smaller than 95, 90, 75 or 50 μm. With reference to FIGS. 1-8, provided is a non-limiting, example oral care product 100 in accordance with an aspect of the invention. Oral care product 100 typically comprises a vessel portion 110 and a cap 120 coupled to the vessel portion 110. Vessel portion 110 is adapted for containing an oral care composition and, in some embodiments, bias the oral care composition toward the cap 120 when the vessel portion 110 is squeezed by a user. Vessel portion 110 may be formed from a polymeric tube, whereby one end of the tube may be sealed by way of a heat press. In some embodiments, however, vessel portion 110 may be formed using extrusion blow-molding techniques. Vessel portion 110 may be formed from or comprise one or more layer formed of polyethylene (low density polyethylene, high density polyethylene, or the like), polyvinyl chloride, ethylene vinyl alcohol, and/or metals (e.g., aluminum). For example, vessel portion 110 may be comprise a plurality of layers, such as two polymeric layers and a metal foil layer (preferably aluminum) positioned therebetween.

Vessel portion 110 is fluidically coupled to cap 120, e.g., by way of a neck portion 112 extending from vessel portion 110. Cap 120 may be configured in any suitable shape that sealably couples to neck portion 112. For example, cap 120 may fit over an exterior portion of neck portion 112. However, neck portion 112 and cap 120 may alternatively be configured such that cap 120 sealably attaches to an interior portion of neck portion 112. Cap 120 may suitably be configured in other shapes, such as, e.g., having a square, rectangular, or triangular cross section.

In some embodiments, one of neck portion 112 and cap 120 includes a protrusion while the other of neck portion 112 and cap 120 includes a recess to receive the protrusion, such that the protrusion and recess hold vessel portion 110 and cap 120 together, while optionally allowing the two portions to rotate about an axis, with respect to each other. The recess may be a groove, such as a spiraled grove or a threading.

In some embodiments, neck portion 112 and cap 120 are configured to allow vessel portion 110 to be detachably coupled to cap 120. In this case, neck portion 112 and cap 120 may be snap-fit together or one of neck portion 112 and cap 120 may include a threaded portion while the other of neck portion 112 and cap 120 may include an internal portion (e.g., a threaded portion) to threadably receive the other portion, such that neck portion 112 and cap 120 form a seal when the two parts are threadably engaged. In accordance with yet other embodiments, neck portion 112 and cap 118 may engage using a lug or an interference-fit structure.

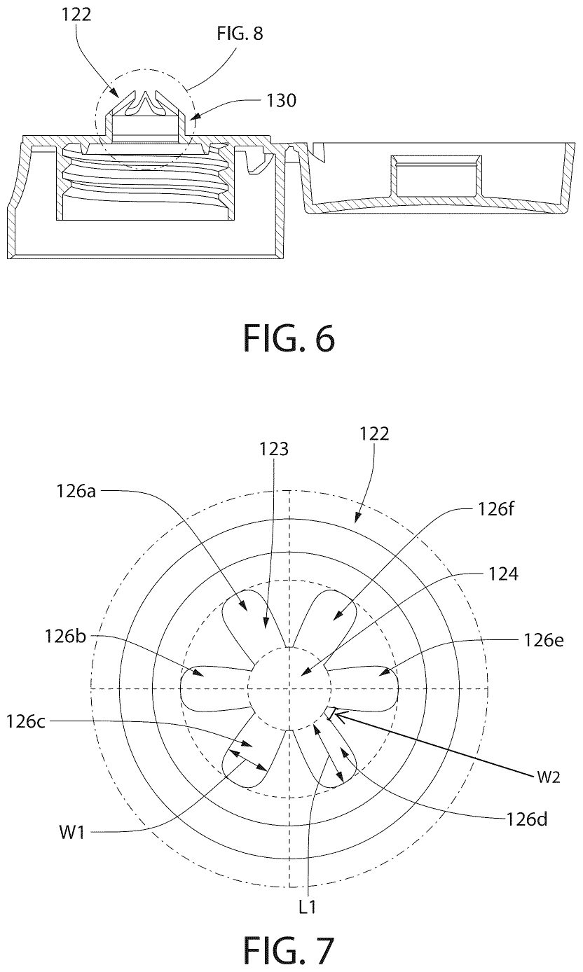

Cap 120 comprises a nozzle 122 adapted for dispensing an oral care composition. Nozzle 122 may be fluidically coupled to cap 120 by way of a channel portion 130. For instance, channel portion 130 may delineate a passageway extending to neck portion 112 of the vessel portion 110 such that an oral care composition disposed in vessel portion 110 can flow to nozzle 122 for dispensing. Although the embodiment in illustrate in FIG. 6 shows depicts nozzle 122 coupled to channel portion 130 of cap 120, in some embodiments nozzle 122 is directed coupled or attached to the base 121 of cap 120.

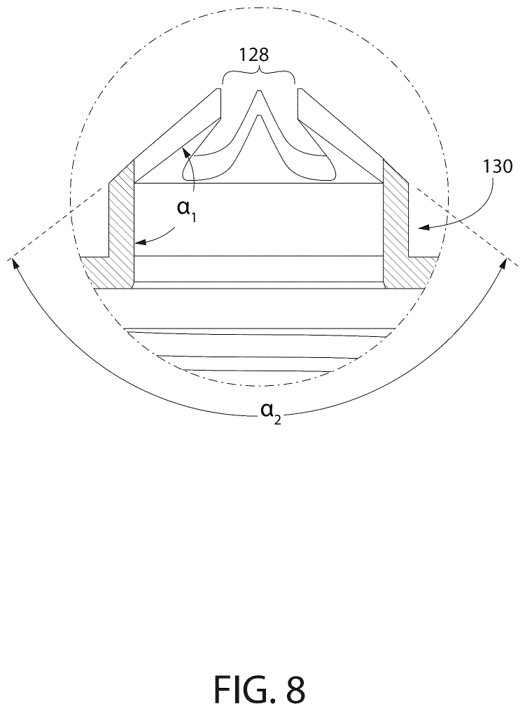

Nozzle 122 may be configured to form a tip 128, e.g., as illustrated in FIGS. 1-8. For instance, nozzle 122 may extend from channel portion 130 radially inwards at an angle α₁ of about 100 to about 170° to form a tip 128. For instance, the nozzle may extend radially inwards, to form a tip 128, at an angle α₁ of about 100 to about 170°, about 120 to about 170°, about 130 to about 170°, about 135 to about 170°, about 140 to about 170°, about 150 to about 170°; from about 100 to about 155°, about 120 to about 155°, about 130 to about 155°, about 135 to about 155°, about 140 to about 155°; from about 100 to about 145°, about 120 to about 145°, about 130 to about 145°, about 135 to about 145°; from about 100 to about 140°, about 120 to about 140°, about 130 to about 140°, about 135 to about 140°; from about 100 to about 135°, about 120 to about 135°, about 130 to about 135°; from about 100 to about 130°, about 120 to about 130°, including any range or subrange thereof, relative to the channel portion 130. As illustrated in FIG. 8, tip 128 may extend from channel portion 130 radially inwards at an angle di relative to the inner surface of channel portion 130, such that tip 128 is raised (e.g., extends outward and/or in a direction away from base 121).

As seen in FIG. 8, nozzle 122 may be configured to preferably form a tip 128 having an internal angle α₂ of about 70 to about 130°. Preferably, the tip forms an internal angle α₂ of about 70 to about 130°, about 70 to about 120°, about 70 to about 110° about 70 to about 105°, about 70 to about 100°, about 70 to about 95°, about 70 to about 90°; from about 80 to about 130°, about 80 to about 120°, about 80 to about 110° about 80 to about 105°, about 80 to about 100°, about 80 to about 95°; from about 90 to about 130°, about 90 to about 120°, about 90 to about 110° about 90 to about 105°, about 90 to about 100°; from about 95 to about 130°, about 95 to about 120°, about 95 to about 110° about 95 to about 105°; from about 100 to about 130°, about 100 to about 120°, about 100 to about 110°; from about 105 to about 130°, about 105 to about 120°, about 105 to about 115°, or any range or subrange thereof.

With Reference to FIG. 7, nozzle 122 delineates an aperture 123 configured to have a central portion 124 and a plurality of legs 126 extending radial from the central portion 124. While the preferred embodiment shown in FIGS. 5 and 7 has an aperture 123 with 6 legs 126a-126f, the aperture 123 of nozzle 122 may be configured to have a plurality of legs 126 comprising from 3 to 9 legs. Preferably, the nozzle 122 is configured to have an aperture 123 having a plurality of legs 126 ranging from 4 to 9 legs, 5 to 9 legs, 6 to 9 legs, 4 to 8 legs, 5 to 8 legs, 6 to 8 legs, 4 to 7 legs, 5 to 7 legs, or 6 to 7 legs. Nozzle 122 may be configured such that the width between two adjacent legs at 0.5 mm from where the two legs connect (W₂) is about 0.1 to about 0.9 mm, e.g., about 0.1 to about 0.7 mm, about 0.1 to about 0.5 mm, about 0.1 to about 0.3 mm; from about 0.2 to about 0.9 mm about 0.2 to about 0.7 mm, about 0.2 to about 0.5 mm; from about 0.4 to about 0.9 mm, about 0.4 to about 0.7 mm, or any range or subrange thereof.

Nozzle 122 may be specifically configured to have a total area of the aperture 123 associated with the plurality of legs (also referred to herein as the leg portion area) that is greater than the area of aperture 123 corresponding to the center portion of the aperture 123 (also referred to herein as the center portion area). In certain embodiments, nozzle 122 is configured to delineate an aperture 123 having a ratio of the center portion area to the leg portion area of about 1:1.5 to about 1:8. For example, the nozzle 122 may be specifically adapted to have a ratio of the center portion area to the leg portion area of about 1:1.5 to about 1:8, about 1:1.5 to about 1:6, about 1:1.5 to about 1:5, about 1:1.5 to about 1:4, about 1:1.5 to about 1:3.5, about 1:1.5 to about 1:3, about 1:1.5 to about 1:2.5; from about 1:2 to about 1:8, about 1:2 to about 1:6, about 1:2 to about 1:5, about 1:2 to about 1:4, about 1:2 to about 1:3.5, about 1:2 to about 1:3; from about 1:2.5 to about 1:8, about 1:2.5 to about 1:6, about 1:2.5 to about 1:5, about 1:2.5 to about 1:4, about 1:2.5 to about 1:3.5, about 1:2.5 to about 1:3; from about 1:3 to about 1:8, about 1:3 to about 1:6, about 1:3 to about 1:5, about 1:3 to about 1:4, about 1:3 to about 1:3.5; from about 1:3.5 to about 1:8, about 1:3.5 to about 1:6, about 1:3.5 to about 1:5, about 1:3.5 to about 1:4; from about 1:4 to about 1:8, about 1:4 to about 1:6, about 1:4 to about 1:5; from about 1:4.5 to about 1:8, about 1:4.5 to about 1:6; from about 1:5 to about 1:8, about 1:5 to about 1:6, including any range or subrange thereof.

Additionally, or alternatively, one or more of the plurality of legs 126 extends from center portion 124 of the aperture 123 by a length L₁ that is greater than the maximum width W₁ of the same leg 126. Preferably, one or more of the plurality of legs 126 is configured to have a ratio of the length L₁ of the leg 126 to the maximum width W₁ of the same leg 126 of about 1:1 to about 1:4, about 1:1.5 to about 1:4, about 1:1.75 to about 1:4, about 1:2 to about 1:4, about 1:2.5 to about 1:4, about 1:3 to about 1:4; from about 1:1 to about 1:3, about 1:1.5 to about 1:3, about 1:1.75 to about 1:3, about 1:2 to about 1:3, about 1:2.5 to about 1:3; from about 1:1 to about 1:2.5, about 1:1.5 to about 1:2.5, about 1:1.75 to about 1:2.5, about 1:2 to about 1:2.5; from about 1:1 to about 1:2.25, about 1:1.5 to about 1:2.25, about 1:1.75 to about 1:2.25; from about 1:1 to about 1:2, about 1:1.5 to about 1:2, about 1:1.75 to about 1:2; from about 1:1 to about 1:1.75, about 1:1.25 to about 1:1.75, including any range or subrange thereof.

In at least one embodiment, the length L₁ of each leg 126a-126f is about the same or is the same as the length L₁ of each other leg 126a-126f. The maximum width W₁ of each leg 126a-126f may also be about the same or the same as the maximum width W₁ of each other leg 126a-126f in a preferred embodiment. Preferably, each of the plurality of legs 126 is configured to have a ratio of the length L₁ of the leg 126 to maximum width W₁ of the same leg 126 within one of the foregoing ranges of ratios. In some preferred embodiment, each of the plurality of legs 126 is configured to have a ratio of the length L₁ of the leg 126 to maximum width W₁ of the same leg 126 that is about the same or the same as each of the plurality of legs 126.

Without being limited to any particular theory, the ratio of the center portion area to the leg portion area, the ratio of the length L₁ of the leg 126 to maximum width W₁ of the same leg 126, the internal angle α₂ forming tip 128 may enhance the preferably mousse rheology and/or mousse aesthetics, which are further discussed below.

In some embodiments, a Maximum Bubble Tensiometer (MBP100 from DataPhysics), is used to measure dynamic surface tension with varying surface age. Those data are fitted with Equation (1)

$$γ ( t ) = {γ}_{m} + \frac{{γ}_{0} - {γ}_{m}}{{1 + ( t / t \text{*)}}^{n}} \qquad ( 1 )$$

where γ₀ is the surface tension of water, which is assumed to be 72.8 mN/m in this report, γₘ is the final surface tension of the sample at infinitely high surface age, t* is the inflection point, and the exponent n represents how fast the curve decays. From the fitted values, surface tension reduction rate (R_{1/2}), which represents the slope at t*, is calculated by Equation (2).

$${R}_{1 / 2} = \frac{{γ}_{0} - {γ}_{m}}{2 t *} \qquad ( 2 )$$

In some embodiments, the oral care composition comprises a surfactant system including an alkyl glucoside in combination with an acyl taurate salt. Suitable alkyl glucosides include C8-C20 alkyl glucosides, such as decyl glucoside, lauryl glucoside, coco-glucoside, and mixtures thereof. Suitable acyl taurate salts include C8-C18 acyl taurates such as sodium methyl cocoyl taurate, sodium methyl lauroyl taurate, potassium cocoyl taurate, and mixtures thereof, wherein the counterion may be selected from sodium, potassium, ammonium, and ethanolamine. In certain embodiments, the weight ratio of alkyl glucoside to acyl taurate is from about 0.5:1 to about 3:1, for example from about 0.8:1 to about 2:1. The composition is substantially free of sulfate-based surfactants (for example, sodium lauryl sulfate, sodium laureth sulfate, ammonium lauryl sulfate), such that the total amount of sulfate-based surfactants is about 0.1 wt. % or less, preferably about 0.05 wt. % or less, more preferably about 0.01 wt. % or less, based on the total weight of the oral care composition. The surfactant system is selected to provide rapid interfacial stabilization as reflected by a dynamic surface tension reduction rate, R½, of at least 0.020 mN/m/ms, for example at least 0.022 mN/m/ms, at least 0.025 mN/m/ms, or at least 0.030 mN/m/ms, when evaluated as a 1% by weight slurry in deionized water at 25±2° C. using a maximum bubble pressure method as described herein. Without being bound by theory, the combination of the alkyl glucoside and acyl taurate salt provides synergistic adsorption kinetics and interfacial packing that facilitates fast surface tension decay, enhanced foamability, and fine, uniform bubble formation in mousse-like nurdles upon dispensing.

In some embodiments, a method of using the dynamic mousse oral care composition comprises dispensing a nurdle of the composition from a vessel through a nozzle onto an oral care implement, such as a toothbrush, and brushing the teeth and oral surfaces for a period of about 30 seconds to about 3 minutes. The composition may be dispensed by manually squeezing the vessel to form a mousse-like nurdle characterized by rapid interfacial stabilization and fine, uniform bubbles, as evidenced by a dynamic surface tension reduction rate (R_{1/2}) of at least 0.020 mN/m/ms when tested as a 1% by weight slurry in deionized water at 25±2° C. The brushing step may include contacting enamel, dentin, and gingival tissues with the mousse to enhance surface coverage and retention, followed by expectoration and optional rinsing. In certain embodiments, the method further comprises applying the composition twice daily, using an amount of about 0.5 g to about 2.0 g per application, and maintaining contact with oral surfaces for at least about 60 seconds to facilitate delivery of active agents (e.g., fluoride) while leveraging the mousse rheology and foamability to improve mouthfeel and user compliance.

In some embodiments, the present disclosure provides a method of treating or preventing erosive tooth demineralization, gingivitis, plaque, and/or dental caries, the method comprising the application to the oral cavity of a subject in need thereof any one of the compositions described herein, e.g., by brushing, for example, one or more times per day.

In other embodiments, the present disclosure provides a method of stabilizing fluoride in an oral care composition using—inter alia—a natural calcium carbonate abrasive system and an sulfate-free surfactant system (e.g., free of sodium lauryl sulfate).

The compositions of the present invention can be administered regularly, such as, for example, one or more times per day. In various embodiments, administering the compositions of the present disclosure to a patient can provide one or more of the following benefits: (i) reduce hypersensitivity of the teeth, (ii) reduce plaque accumulation, (iii) reduce or inhibit demineralization and promote remineralization of the teeth, (iv) inhibit microbial biofilm formation in the oral cavity, (v) reduce or inhibit gingivitis, (vi) promote healing of sores or cuts in the mouth, (vii) clean the teeth and oral cavity, (viii) reduce erosion, (ix) whiten teeth; (x) reduce tartar build-up, and/or (xi) promote systemic health by, e.g., reducing the potential for systemic infection via the oral tissues. The disclosure further provides compositions for use in any of the above methods.

The citation of any document is not an admission that it is prior art with respect to any invention disclosed or claimed herein or that it alone, or in any combination with any other reference or references, teaches, suggests or discloses any such invention. While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example 1

Several exemplary oral care compositions are prepared in the form of a mousse in accordance with certain aspects of the present invention. Tables 1 and 2 (below) describe the ingredient listings for these exemplary compositions (Ex. A to Ex. D) and three comparative compositions (Comp. Ex. 1 to Comp. Ex. 3).

TABLE 1

| | Ex. A | Ex. B | Ex. C | Ex. D | Comp. Ex. 1 |
|---|---|---|---|---|---|
| US INCI Compound Name | Wt. % | | | | |
| Glycerin | 34.7 | 19.8 | 20 | 20 | 20 |
| Water | ~30 | ~40 | 36-39 | 36.14 | 34.43 |
| Hydrated Silica | 18.9 | 2.3 | | | |
| Thickening Silica | | | 2.5 | 2.5 | |
| Crystalline Silica | | 0.1 | | | |
| Natural Calcium Carbonate | | 29.9 | 30 | 31 | |
| Precipitated Calcium Carbonate | | | | | 35 |
| Sodium Carbonate and Sodium | | 0.5 | 0.5 | 0.5 | 0.4 |
| Bicarbonate | | | | | |
| Potassium Carbonate | 0.01 | 0.01 | | | |
| Sodium Methyl Cocoyl Taurate | 1 | 1 | 1 | 1 | |
| Cocamidopropyl Betaine | 0.4 | 0.4 | 1.25 | 1.25 | 1.25 |
| Lauryl Glucoside | | | 3 | 3 | |
| Sodium Lauryl Sulfate | | | | | 1.62 |
| D-Glucopyranose, Oligomeric, C₁₀₋₁₆- | 1.7 | 1.7 | | | |
| Alkyl Glycosides | | | | | |
| Xanthan Gum | 0.5-1 | 0.4 | 0.02-0.05 | 0.4 | 0.4 |
| Acrylates/C₁₀₋₃₀ Alkyl Acrylate | 0.2 | 0.2 | 0.2 | 0.2 | 0.2 |
| Crosspolymer (e.g., Carbopol ETD 2020 | | | | | |
| NF) | | | | | |
| Carbomer (e.g., Carbopol 956) | 0.2 | 0.2 | 0.2 | 0.2 | 0.2 |
| Sodium Carboxymethylcellulose | | 0.7 | 0.7 | 0.9 | |
| Tetrasodium Pyrophosphate | 0.5 | 0.4 | 0.4 | 0.5 | 0.6 |
| Sodium Fluoride and/or Sodium | 0.24 | 0.75 | 0.76 | 0.76 | 1.1 |
| Monofluorophosphate | | | | | |
| Sodium Sulfate | 0.4 | 0.05 | | | |
| Sweetening Agents (Rebaudioside A, | 10.3 | 0.3 | | 1.25 | 0.27 |
| Sodium Saccharin, Erythritol, and/or | | | | | |
| Xylitol | | | | | |
| Flavoring Agents | 0.8 | 0.8 | 0.8 | | 1 |
| Benzyl Alcohol | | 0.4 | 0.4-0.6 | 0.4 | 0.4 |
| Sodium Chloride | 0.09 | 0.09 | | | |

TABLE 2

| | Comp. Ex. 2 | Comp. Ex. 3 |
|---|---|---|
| Ingredient | Wt. % | |
| Dicalcium Phosphate Dihydrate | | 44.00 |
| Water | | 25.75 |
| Glycerin | 11.00 | 22.00 |
| 29% Sodium Lauryl Sulfate | | 4.91 |
| Sodium Carboxymethyl Cellulose | | 1.20 |
| Flavor | | 0.95 |
| Sodium Monofluorophosphate | | 0.76 |
| Tetrasodium Pyrophosphate | | 0.25 |
| Sodium Saccharin | | 0.18 |
| Sorbitol - Non-crystal - 70% Soln. | 40.00 | |
| Synthetic High Cleaning Silica | 12.00 | |
| Water | 11.96 | |
| Xylitol | 5.00 | |
| Limestone | 5.00 | |
| Erythritol | 5.00 | |
| Synthetic Thickening Silica | 4.00 | |
| Cocamidopropyl Betaine | 1.37 | |
| Xanthan Gum | 1.12 | |
| Flavor | 1.00 | |
| Titanium Dioxide | 0.60 | |
| Sodium Cocoyl Glutamate | 0.50 | |
| Potassium Sorbate | 0.50 | |
| Zinc Citrate Trihydrate | 0.50 | |
| Coconut Oil | 0.25 | |
| Rebaudioside A 98% | 0.15 | |
| Tear Tree (Melaleuca Alternifolia) Oil | 0.05 | |
| Total Components | 100.00 | 100.00 |

### Example 2

Fluoride stability for two exemplary compositions of the present invention (Ex. C. and Ex. D) and a comparative composition (Comp. Ex. 1) is evaluated. The compositions are evaluated under accelerated aging conditions to determine the amount of soluble fluoride remaining at various time points up to three (3) months at 40° C. The results of these evaluations are described below in Table 3.

TABLE 3

| | Fluoride Level | | | |
|---|---|---|---|---|
| Formula | Initial | 1 Mo/40° C. | 2 Mo/40° C. | 3 Mo/40° C. |
| Comp. Ex. 1 w/PCC | 1037 | 990 | 780 | 680 |
| Ex. C | 1040 | 1000 | 1030 | 990 |
| Ex. D | 980 | 1010 | 830 | 970 |

PCC = precipitated calcium carbonate

### Example 3

Dynamic surface tension measured by maximum bubble pressure is a measure of how surfactants quickly stabilize when the new surface is created, for example, during foaming. The faster stabilization indicates that it is easy to create foam. Here, dynamic surface tension of four toothpastes is measured—three comparative compositions (Comp. Ex. 1 to Comp. Ex. 3) and one exemplary composition of the present invention (Ex. C). As illustrated below, an exemplary composition of the present invention—having a unique surfactant system—is the most effective in stabilizing surface tension.

### Compositions Evaluated:

1. Comp. Ex. 1
2. Comp. Ex. 2
3. Ex. C
4. Comp. Ex. 3
   1% slurry in DI water.

Using Maximum Bubble Tensiometer (MBP100 from DataPhysics), dynamic surface tension was measured with varying surface age. Those data are fitted with Equation (1)

$$γ ( t ) = {γ}_{m} + \frac{{γ}_{0} - {γ}_{m}}{{1 + ( t / t \text{*)}}^{n}} \qquad ( 1 )$$

where γ₀ is the surface tension of water, which is assumed to be 72.8 mN/m in this report, γₘ is the final surface tension of the sample at infinitely high surface age, t* is the inflection point, and the exponent n represents how fast the curve decays. From the fitted values, surface tension reduction rate (R_{1/2}), which represents the slope at t*, is calculated by Equation (2).

$${R}_{1 / 2} = \frac{{γ}_{0} - {γ}_{m}}{2 t *} \qquad ( 2 )$$

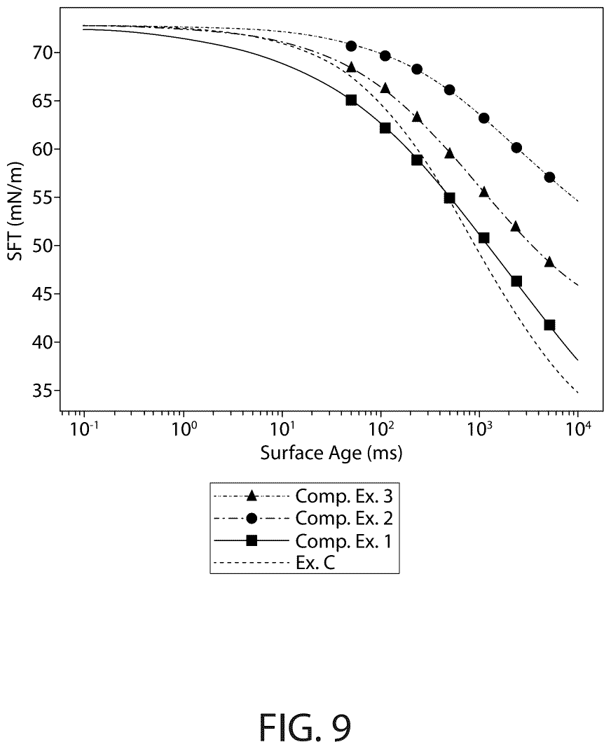

As shown in FIG. 9, dynamic surface tension is calculated in terms of surface age. The circles represent the measured data, and the solid lines are the fitted lines using Equation (1). The data is fitted with Equation (1), and the fitting parameters are tabulated in Table 4 (below).

TABLE 4

| | γₘ (mN/m) | t* (ms) | n | R_{1/2} (mN/m/ms) |
|---|---|---|---|---|
| Comp. Ex. 1 | 22.31 | 1,857.67 | 0.47 | 0.0136 |
| Comp. Ex. 2 | 47.00 | 2,491.76 | 0.64 | 0.0052 |
| Comp. Ex. 3 | 39.67 | 961.17 | 0.63 | 0.0172 |
| Ex. C | 27.68 | 885.92 | 0.70 | 0.0255 |

The results provide that higher R_{1/2} relates to faster foaming because fast decrease in surface tension over surface age implies easier surface stabilization by surfactants. Among the four samples evaluated, the exemplary composition of the present invention (Ex. C) delivered the highest R_{1/2}, which indicates its superiority in foaming capacity. Compared to Comp. Ex. 2, Ex. C contains higher amounts of lauryl glucoside and sodium methyl cocoyl taurate, which contributes to the higher R_{1/2} observed. Surprisingly, the exemplary composition of the present invention also showed higher R_{1/2} than Comp. Ex. 1 and Comp. Ex. 3, which both contain sodium lauryl sulfate-based surfactant systems.

### Example 4

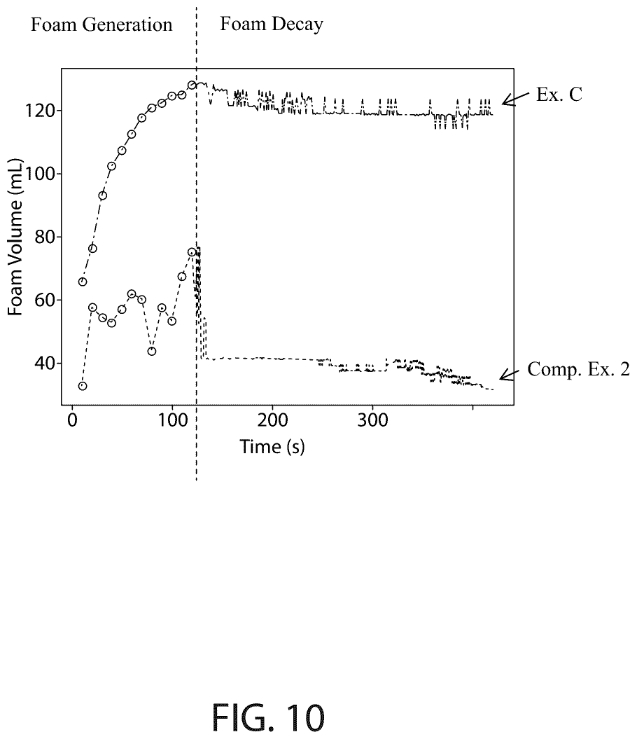

Foam generation and foam decay of an exemplary composition of the present invention (Ex. C) and a comparative composition (Comp. Ex. 2) is evaluated using a Dynamic Foam Analyzer (DFA). DFAs are available from Kruss Scientific (e.g., DFA100). Foam formation and decay are dynamic processes. Foam formation represents foam volume as a function of time, while foam decay measures the foam volume in a resting state. The results of the evaluations performed on an exemplary composition of the present invention (Ex. C) and the comparative composition (Comp. Ex. 2) are described in FIG. 10.

As illustrated by the data described in FIG. 10, the exemplary composition of the present invention generates significantly more foam than the comparative composition. The foam decay observed with the exemplary composition of the present invention is also considerably less.

### Example 5

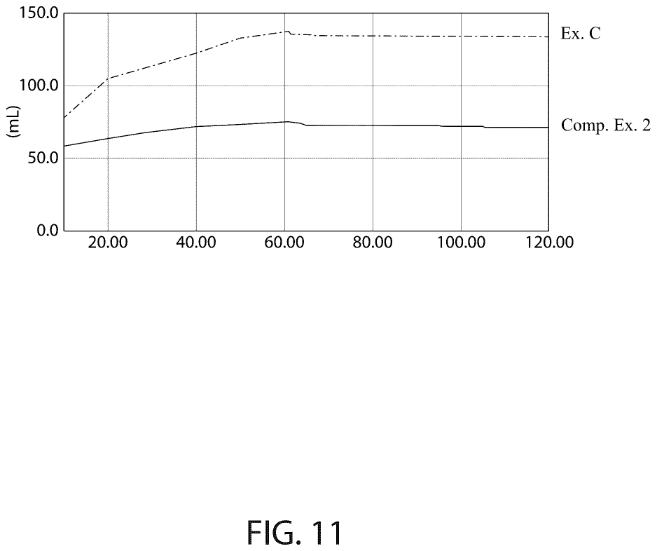

Foam generation by an exemplary composition of the present invention (Ex. C) and a comparative composition (Comp. Ex. 2) are evaluated in accordance with conventional methods known to those skilled in the art using a SITA Foam Tester. The results of the evaluations performed on an exemplary composition of the present invention (Ex. C) and the comparative composition (Comp. Ex. 2) are described in FIG. 11.

### Example 6

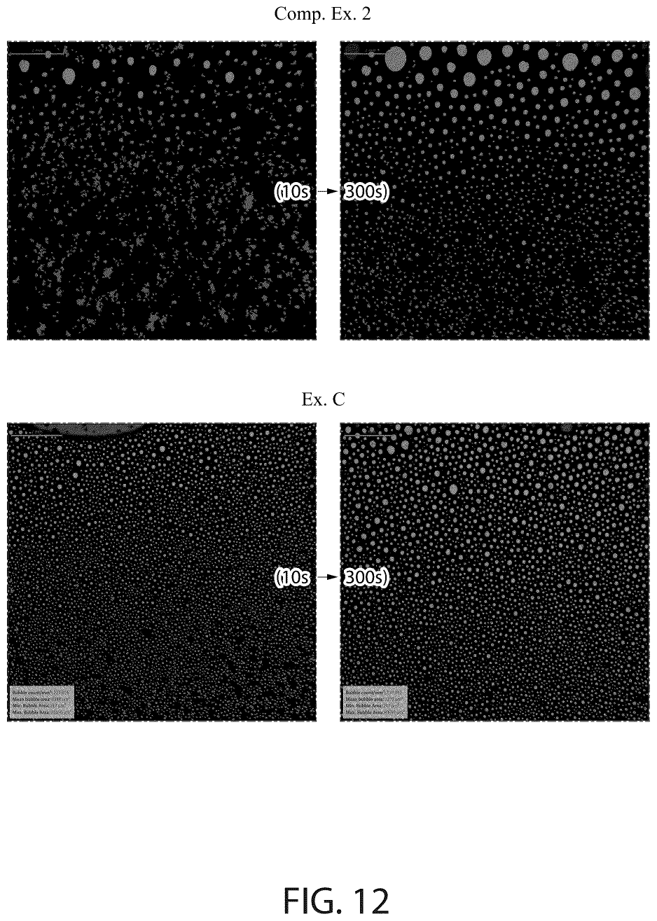

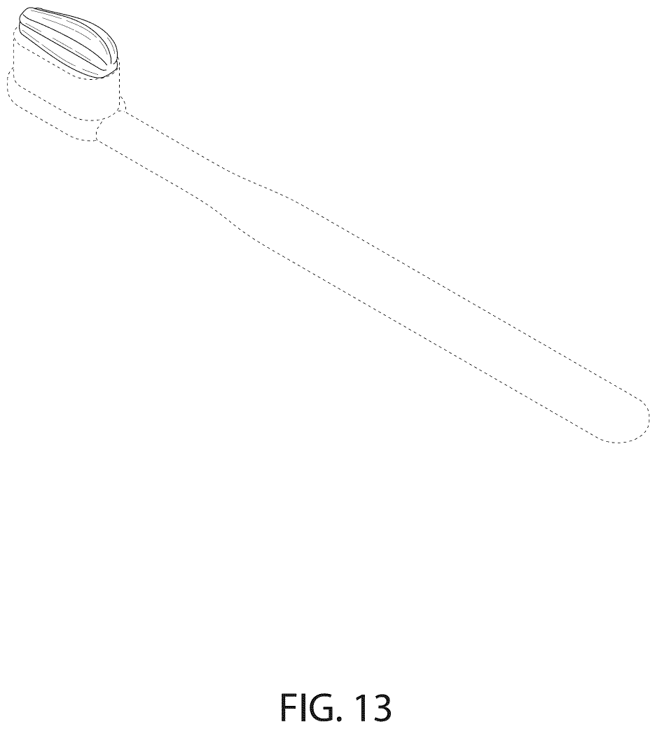

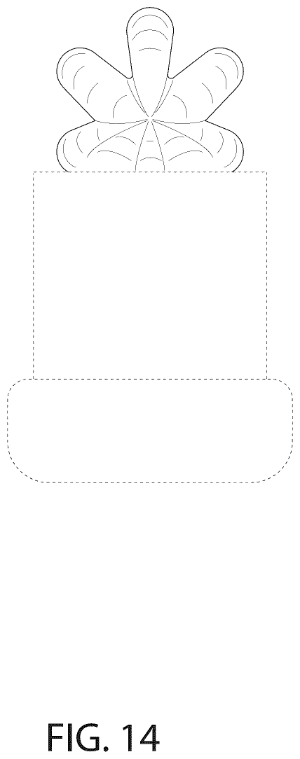

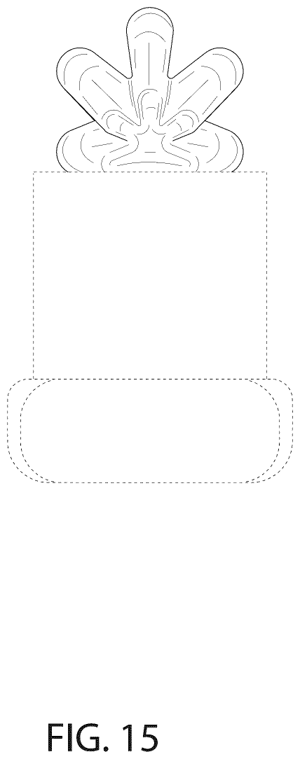

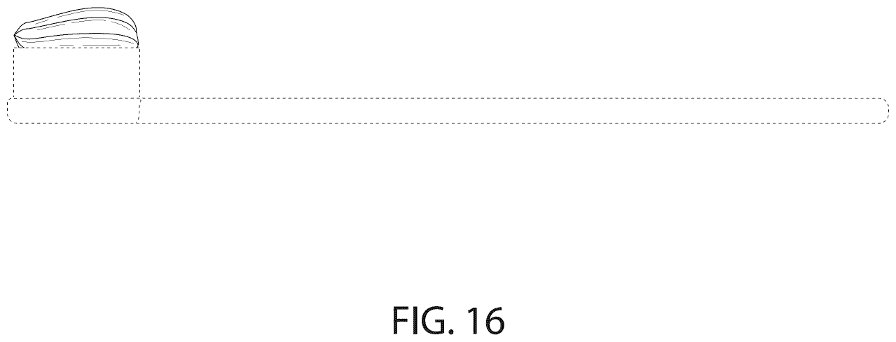

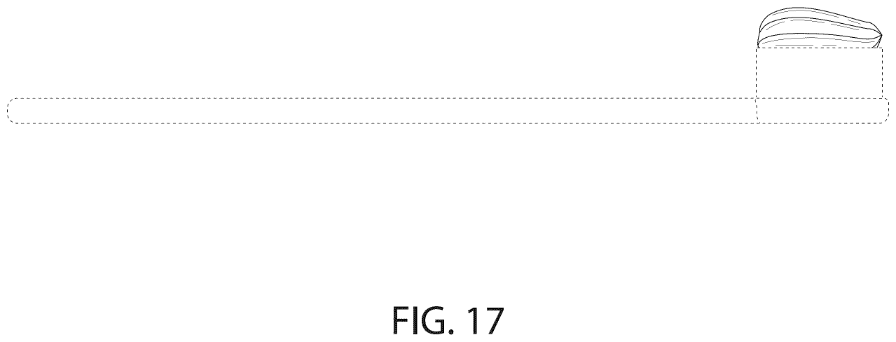

A Dynamic Foam Analyzer (DFA) is used to evaluate the bubble size and uniformity of the foam created by an exemplary composition of the present invention (Ex. C) and a comparative composition (Comp. Ex. 2). As illustrated by the data depicted in FIG. 12, the exemplary composition of the present invention provided smaller and more uniformly sized bubbles than the comparative composition. Smaller bubble size is indicative of a more stable foam which often correlates to better mouthfeel. The bubble comparison is provided below in Table 5.

TABLE 5

| | |
|---|---|
| Radius | Diameter |

| | |
|---|---|
| Sample | μm |

| | | |
|---|---|---|
| Ex. C | 47 | 94 |
| Comp. Ex. 2 | 57 | 114 |

It is understood, therefore, that this invention is not limited to the particular embodiments disclosed but is intended to cover all modifications which are within the spirit and scope of the invention as defined by the appended claims; the above description; and/or shown in the attached drawings.

What is claimed is:

1. An oral care product comprising:
an oral care package comprising a vessel portion and a cap coupled to the vessel portion, the cap comprising a nozzle; and
an oral care composition disposed in the vessel portion, the oral care composition comprising:
from about 10 to about 43 wt. % of an abrasive;
a surfactant system comprising:
an amphoteric surfactant, and
a nonionic surfactant; and
a thickening system comprising:
a natural gum, and
an acrylic acid polymer,
wherein the oral care composition has a stress drop of about 18 to about 60% and an adhesive force of about 60 to about 200 gram-force, and
wherein the oral care product is free of propellants and all weight percentages are based on the total weight of the oral care composition.

2. The oral care product according to claim 1, wherein the nozzle delineates an aperture configured to have a central portion and a plurality of legs extending radially from the central portion.

3. The oral care product according to claim 2, wherein the plurality of legs comprises 3 to 9 legs.

4. The oral care product according to claim 2, wherein the plurality of legs comprises 5 to 7 legs.

5. The oral care product according to claim 2, wherein the aperture has a ratio of the center portion area to the leg portion area of about 1:1.5 to about 1:8.

6. The oral care product according to claim 2, wherein each of the plurality of legs extends from center portion by a length that is greater than the maximum width of the same leg.

7. The oral care product according to claim 6, wherein the length of each of the plurality of legs is about the same as the length of each other leg of the plurality of legs.

8. The oral care product according to claim 6, wherein the width of each of the plurality of legs is about the same as the width of each other leg of the plurality of legs.

9. The oral care product according to claim 6, wherein one or more of the plurality of legs is configured to have a ratio of the length of the leg to maximum width of the same leg of about 1:1 to about 1:4.

10. The oral care product according to claim 6, wherein each of the plurality of legs is configured to have a ratio of the length of the leg to maximum width of the same leg of about 1:1 to about 1:4.

11. The oral care product according to claim 1, wherein the nozzle is coupled to a channel portion extending from the cap.

12. The oral care product according to claim 11, wherein the nozzle extends from the cannel portion radially inwards at an angle of about 120 to about 155°.

13. An oral care composition comprising:
from about 10 to about 43 wt. % of an abrasive, the abrasive comprising an alkali metal carbonate, an alkali earth metal carbonate, an insoluble phosphate, or a combination of two or more thereof;
a surfactant system;
a thickening system comprising:
from about 0.01 wt. % to about 0.5 wt. % of a natural gum, and
an acrylic acid polymer, wherein the natural gum and the acrylic acid polymer are present in a weight ratio of the natural gum to the polyacrylic acid polymer of about 5:1 to about 1:5;
from about 15 to about 44 wt. % of a polyol; and
from about 20 to about 50 wt. % of water,
wherein the oral care composition has a stress drop of about 18 to about 60% and an adhesive force of about 60 to about 200 gram-force, and
wherein the oral care composition is free of propellants and all weight percentages are based on the total weight of the oral care composition.

14. The oral care composition according to claim 13, wherein the abrasive comprises calcium carbonate, calcium bicarbonate, or a combination of two or more thereof, wherein the total amount of calcium carbonate and/or calcium bicarbonate comprises about 80 wt. % or more of the total amount of the abrasive agent in the oral care composition.

15. The oral care product according to claim 1, wherein the oral care package provides a nurdle having a dynamic surface tension reduction rate (R_{1/2}) of at least 0.020 mN/m/ms, when tested as a 1% by weight slurry in deionized water at 25±2° C.

16. The oral care product according to claim 1, wherein the oral care package provides a nurdle comprising bubbles with an average diameter of less than 100 μm, optionally less than 95 μm, or less than 90 μm.

17. The oral care product according to claim 16, wherein the oral care package provides a nurdle comprising bubbles with an average diameter of about 95 μm.

18. The oral care product according to claim 16, wherein the oral care package provides a nurdle comprising bubbles that are substantially homogenous.

19. The oral care product of claim 1, wherein the composition is free of sulfate-based surfactants.

20. An oral care composition comprising:
a surfactant system comprising: an alkyl glucoside and an acyl taurate salt; and
an abrasive system comprising a natural calcium carbonate;
wherein the composition is substantially free of sulfate-based surfactants; and
wherein the composition exhibits a dynamic surface tension reduction rate (R_{1/2}) of at least 0.020 mN/m/ms, when tested as a 1% by weight slurry in deionized water at 25±2° C.
